SEQUENCE LISTING (11 sequences)

SEQ ID NO: 1 - protein, 805 aa, Homo sapiens [sequence omitted]

SEQ ID NO: 2 - DNA, 1986 bp, synthetic construct
  source 1..1986: mol_type = other DNA
cgatgctcacggatccgccaccatgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggctccaatgtgtttcaaacgcgtgccggctgtcgaattcatcgtcgacg

SEQ ID NO: 3 - DNA, 1957 bp, synthetic construct
  source 1..1957: mol_type = other DNA
ggcagcaatggaattctgtttcagacgcgtgccggctgcctgattggagccgaacacgtgaacaacagctacgagtgcgacatccctatcggagccggcatctgtgccagctaccagacacagacaaacagccccagacgggccagatctgtggccagccagagcatcattgcctacaccatgtctctgggcgccgagaacagcgtggcctacagcaacaactctatcgctatccccaccaacttcaccatcagcgtgaccaccgagatcctgcctgtgtccatgaccaagaccagcgtggactgcaccatgtacatctgcggcgatagcaccgagtgcagcaacctgctgctgcagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgaccctggccgacgccggcttcatcaagcagtatggcgattgcctgggcgacattgccgccagggatctgatttgcgcccagaagtttaacggcctgaccgtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggcgtgacccagaacgtgctgtacgagaaccagaagctgatcgccaaccagttcaacagcgccatcggcaagatccaggacagcctgagcagtacagccagcgctctgggaaagctgcaggacgtggtcaaccagaatgcccaggctctgaacaccctggtcaagcagctgagcagcaacttcggcgccatcagcagcgtgctgaacgacatcctgagccgcctggataaggtggaagccgaggtgcagatcgaccggctgattacaggcagactgcagagcctgcagacctacgtgacacagcagctgatcagagccgccgagattagagcctctgccaatctggccgccaccaagatgtctgagtgtgtgctgggccagagcaagagagtggacttttgcggcaagggctaccacctgatgagcttccctcagtctgctcctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgactcagcggaacttctacgagccccagatcatcaccaccgacaacaccttcgtgtccggcaactgcgacgtcgtgatcggcatcgtgaacaataccgtgtacgaccctctgcagcccgagctggacagcttcaaagaggaactggacaagtacttcaagaaccacacaagccccgacgtggacctgggcgatatcagcggaatcaatgccagcgtcgtgaacatccagaaagagatcgacagactgaacgaggtggccaagaacctgaacgagagcctgatcgacctgcaagagctggggaagtacgagcagtatatcaagtggccctggtacatctggctgggctttatcgccggcctgattgccatcgtgatggtcacaatcatgctgtgctgcatgaccagctgttgcagctgcctgaagggctgctgtagctgtggctcctgctgcaagttcgacgaggacgattctgagcccgtgctgaagggcgtgaagctgcactacacatacccctacgacgtgcccgactacgcctaactcgagcgtcagcgta

SEQ ID NO: 4 - DNA, 2418 bp, synthetic construct
  source 1..2418: mol_type = other DNA
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 5 - protein, 1282 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTYPYDVPDYA

SEQ ID NO: 6 - protein, 1254 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 7 - protein, 1263 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCYPYDVPDYA

SEQ ID NO: 8 - protein, 1254 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 9 - protein, 748 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSHHHHHHHH

SEQ ID NO: 10 - protein, 879 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHHHH

SEQ ID NO: 11 - protein, 879 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCAPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHHHH

(19) United States
(12) Patent Application Publication
(10) Pub. No.: US 2024/0218392 A1
(43) Pub. Date: Jul. 4, 2024

(54) VECTORED PROPHYLAXIS OF SARS-COV-2

(71) Applicant: New York University, New York, NY (US)

(72) Inventors: Nathaniel R. Landau, New York, NY (US); Takuya Tada, New York, NY (US)

(21) Appl. No.: 18/401,213

(22) Filed: Dec. 29, 2023

Related U.S. Application Data
(60) Provisional application No. 63/436,279, filed on Dec. 30, 2022.

(51) Int. Cl. C12N 15/86; A61P 31/14; C12N 9/48

(52) U.S. Cl. CPC C12N 15/86; A61P 31/14; C12N 9/485; C12Y 304/17023; C07K 2317/526; C07K 2319/30; C12N 2740/15043; C12N 2740/15071; C12N 2750/14143; C12N 2750/14171

(57) ABSTRACT

Provided are composition and methods for use in prophylaxis or therapy of coronavirus infections. The compositions include an angiotensin-converting enzyme 2 (ACE2) ectodomain and a segment of an immunoglobulin Fc that is not an intact Fc region, and expression vectors encoding the same. The expression vectors include viral vectors.

### CROSS REFERENCE TO RELATED APPLICATIONS

This application claims priority to U.S. provisional application No. 63/436,279, filed Dec. 30, 2022, the entire disclosure of which is incorporated herein by reference.

### STATEMENT REGARDING FEDERALLY SPONSORED RESEARCH

This invention was made with government support under grant nos. DA046100 and Al122390 awarded by the National Institutes of Health. The government has certain rights in the invention.

### SEQUENCE LISTING STATEMENT

The instant application contains a Sequence Listing which is submitted in .xml format and is hereby incorporated by reference in its entirety. Said .xml file is named “058636_00672_Sequence.xml”, was created on Dec. 18, 2023, and is 25,079 bytes in size.

### FIELD

The present disclosure relates to compositions and methods for prophylaxis or therapy of Coronavirus infections. The compositions comprise a soluble ACE2 receptor as a component of a fusion protein that also comprises a segment of an Fc region of an antibody. The disclosure includes expression vectors that encode and express the described fusion protein and uses thereof. The expression vectors include viral vectors, such as adeno viral vectors, such as an adeno-associated viral vector.

### RELATED INFORMATION

Monoclonal antibody therapy had been highly successful for the treatment of severe COVID-19, decreasing hospitalization and deaths but has been largely sidelined by the extraordinarily rapid appearance of viral variants that escape neutralization. The first Omicron variant, BA.1, contained 34 mutations in the S1 spike protein, most of which were within or close to the spike protein receptor binding domain that resulted in escape from most of the therapeutic mAbs. The Regeneron REGN-COV2 cocktail, a cocktail of REGN10933 and REGN10987 mAbs, and the Lilly LY-CoV555 potently neutralize the earlier variants of concern (alpha, beta, gamma and delta) but their IC50 against the Omicron BA.1 is greatly increased (Liu et al., 2022; Planas et al., 2021; VanBlargan et al., 2022). The more recent Vir/GSK VIR-7831 (Sotrovimab) was thought to maintain neutralizing activity against the BA.1 and BA.2 variants was decreased 10.5- and 340-fold, respectively(Baum et al., 2020; Hansen et al., 2020). Lilly LY-CoV1404 was the only mAb that retained neutralizing titer against BA.1 and BA.2 but newer, yet further mutated, Omicron variants BA.4/5 have escaped this mAb. As the virus continues to evolve additional variants, it will be challenging to develop mAbs from which the virus cannot escape. The rapidity of virus evolution also presents a challenge to vaccine design. While the vaccine-elicited antibody response is highly polyclonal, variants retain a degree of escape even from the bivalent mRNA booster vaccines the encode BA.1.

Receptor decoys offer the possibility of therapeutics that retain efficacy against current and future variants. While viral spike proteins tend to be highly mutable in response to pressure from neutralizing antibodies, they need to retain high affinity binding to their receptor and thus, soluble forms of the receptor are likely to maintain neutralizing activity against even highly mutated spike proteins. Receptor decoys were first developed to treat HIV infection. A receptor decoy consisting of the cell surface-exposed ectodomain of CD4 fused to an immunoglobulin Fc domain was found to potently neutralize HIV in vitro by binding to the viral gp120 surface glycoprotein. Early clinical trials showed no efficacy (Hodges et al., 1991) (Gershon, 1996) but more recently, the concept was revived and showed that an enhanced eCD4-Ig protected rhesus macaques from multiple challenges with SIV (Gardner et al., 2015).

The concept of vectored immunoprophylaxis was first proposed as an approach to establish protection against HIV infection by the vectored expression of a broadly neutralizing antibody (Johnson et al., 2009) and has since been shown to be effective in nonhuman primate models with AAV vectors expressing neutralizing antibodies or a receptor decoy. An AAV vector-expressed neutralizing antibody was shown to suppress virus load in experimentally infected rhesus macaques (Gardner et al., 2015) and an AAV vector expressing enhanced soluble CD4 protected macaques from SIV infection (Spitsin et al., 2020). However, there remains an ongoing need to provide new vectored approaches for prophylaxis and treatment of coronavirus infections. The present disclosure is pertinent to this need.

### SUMMARY

The disclosure demonstrates, among other embodiments, use of an adeno-associated viral approach to establish long-term prophylaxis against SARS-COV-2. AAV2.retro and AAV6.2 vectors as described further below were generated and express a highly potent ACE-2 decoy receptor fused to a single domain of an immunoglobulin Fc domain. Administration of decoy-expressing AAV vectors by intranasal or intramuscular routes to ACE-2 transgenic mice protected them against high-titered intranasal infection, preventing the appearance of detectable virus. The protection was durable and active against a wide range of SARS-COV-2 variants including omicrons BA.1, BA.2, BA.5 and XBB1.5. The therapy was also effective when administered post-infection with kinetics similar to that of monoclonal antibody or the recombinant decoy protein resulting in a rapid decrease in virus load. Prophylactic and therapeutic approaches are thus provided. In non-limiting embodiments the described compositions and methods are expected to be suitable for use with, for example, immunocompromised individuals for whom vaccination is not practical, or to rapidly establish immunity to infection. Unlike monoclonal antibody therapy, the approach is expected to remain effective with continued evolution of the virus.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1. AAV-ACE2mb prevents SARS-COV-2 infection in cell culture. (A) The domain structure of full-length ACE2 is shown above with the ectodomain, transmembrane (TM) and cytoplasmic domain (IC). The structure of the decoy consisting of the ACE2 ectodomain, human IgG-CH3 and carboxy-terminal His tag. The ACE2 domain contains three high affinity mutations, and a H345A mutation in the ACE2 peptidase catalytic site. (B) A549.ACE2 and CHME3.ACE2 cells were transduced with a 5-fold serial dilution of AAV2.retro and AAV6.2 decoy vectors and then challenged with ancestral D614G Omicron 2.1, 2.2, 2.75, BA.4/5 and BQ1 spike protein-pseudotyped lentiviral vectors. Luciferase activity was measured 2 days post-infection. The curves indicate the infectivity based on luciferase activity normalized to mock vector-transduced cells. Each measurement is shown as the average of duplicates. The table shows ID50 calculated from the curves shown. (C) 293T.ACE2 and A549.ACE2 cells were transduced with AAV2.retro or AAV6.2-ACE2.1mb at MOI=0.5. Three days post-infection, secreted decoy protein in the supernatant was pulled-down on NTA beads and bead-bound decoy was detected on an immunoblot probed with His-tag antibody. Pure recombinant decoy protein is shown at right as a standard and was used to determine the amount of protein pulled-down, which is shown below each lane as micrograms decoy pulled-down from 1 ml of culture supernatant. Shown at the right is the Decoy protein in the cell lysates with GAPDH as a loading control. (D) ACE2-A549 and ACE2-CHME3 cells were transduced with AAV2.retro-GFPNLuc, AAV6.2-GFPNLuc, AAV2.retro-ACE2.1mb, AAV6.2-ACE2.1mb at MOI=0.5. Two days post transduction, the cells were infected with SARS-COV-2 WA1/2020 (MOI=0.01). The cultures were lysed 2 days post-infection and RNA was prepared. Viral RNA copy numbers were determined by RT-qPCR. (E) hsABci cells were transduced with AAV2.retro-GFPNLuc, AAV6.2-GFPNLuc, AAV2.retro-ACE2.1mb, AAV6.2-ACE2.1mb at MOI=0.5. Two days post transduction, the cells were infected with SARS-COV-2 WA1/2020 (MOI=0.01). The cultures were lysed 2 days post-infection and RNA was prepared. Viral RNA copy numbers were determined by RT-qPCR.

FIG. 2. Prophylactic and therapeutic use of AAV vector. (A) The experimental scheme for testing decoy prophylaxis is diagrammed. K18 hACE2-Tg and Balb/c mice were treated by i.v. injection or i.n. instillation with AAV2.retro, AAV6.2 decoy-expressing vector or control GFP-expressing vector (1×10¹² vg). Three days post-treatment, the K18 hACE2 Tg mice were challenged with WA1/2020 SARS-COV-2 and the BALB/c mice were challenged with Omicron BA.1, BA.2 or BA.5 (1×10⁴ PFU). Viral RNA copies were quantified 2 days (Omicron) or 3 days (WA1/2020) post-infection. (B) Mice were treated with decoy-expressing or control GFP-expressing AAV2.retro or AAV6.2 and challenged with WA1/2020 days later. Viral RNA copies in the lungs were quantified 3-dpi. (C) H&E stained lung sections from control and AAV2.retro and AAV6.2-decoy and SARS-CoV-2 infected mice are shown on the left (20×, scale bars 50 μm) and with the boxed area enlarged on the right. (D) Mice were treated with decoy-expressing vectors on day 0 and challenged with WA1/2020 on day 3. Body weight was measured daily. As controls, the mice were infected with WA1/2020 but not treated with AAV vector or treated with AAV vector but not infected with WA1/2020. (E) BALB/c mice were treated with decoy-expressing or GFP-expressing control AAV2.retro and AAV6.2 vectors and then infected 3 days later with Omicron BA.1, BA.2, or BA.5. Viral RNA copies in the lungs were quantified 2-dpi. (F) Mice were administered decoy-expressing AAV vectors i.m. or i.v. and challenged 3-dpi with WA1/2020 SARS-COV-2. Infected but untreated and uninfected/untreated mice are included as controls. (G) Therapeutic use of the decoy-expressing AAV vectors was tested as diagrammed (top). hACE2 K18 Tg (n=4) were infected with WA1/2020 SARS-COV-2 (1×10⁴ PFU) and then treated with decoy-expressing AAVs at time-points up to 48 hours post-infection. Viral RNA in the lung was quantified 3-dpi (bottom). As controls the mice were untreated (Ctr) or uninfected. Confidence intervals are shown as the mean±SD. *** P≤0.001, **** P≤0.0001. The experiment was done twice with similar results.

FIG. 3. Durable vectored prophylaxis by decoy-expressing AAV vectors. (A) Balb/c mice (n=3) were injected i.n. with decoy-luciferase fusion protein-expressing AAV2.retro or AAV6.2 (1×10¹² vg). Luciferase activity was visualized by live imaging over 30 days at the indicated time points. Representative images of a mouse from each group are shown. (B) Luciferase activity in lung tissue homogenates from mice treated with the decoy-luciferase expressing AAV vectors (n=2) was measured over the time course. (C) The experimental scheme to test the durability of AAV vectored prophylaxis is diagrammed (top). hACE2 K18 Tg (n=4) were injected with AAV decoy. At 1-, 2-, 3-dpi, the mice were challenged with SARS-COV-2 (1×10⁴ PFU) and viral RNA in the lungs was quantified. The results are shown as a histogram (bottom). Infected/untreated (Ctrl) and untreated/uninfected controls are shown.

FIG. 4. Long-term vectored prophylaxis by decoy-expressing lentiviral vector. (A) A549.ACE2 and CHME3.ACE2 cells were transduced with a 5-fold serial dilution of pLenti-ACE2.1mb and then challenged with D614G, Omicron BA.1, BA.2, 2.75, BA.4/5 and BQ.1 spike protein-pseudotyped lentiviral vectors. Luciferase activity was measured 2 days post-infection (above). The curves indicate infectivity based on luciferase activity normalized to mock vector-transduced cells. Measurements are the average of duplicates. ID50s calculated from the curves are shown in the table (below). (B) Structure of lentiviral vector and experimental scheme are shown. hACE2-Tg mice or Balb/c were injected with lentiviral vector (5×10⁶ IU) i.p., i.v., or i.n. injection. One week later, the mice were challenged with 1×10⁴ PFU of SARS-COV-2 (hACE2-Tg) or Omicron (Omicron). (C) Mice were administered decoy-expressing lentivirus i.v. or i.n. and after 7 days, challenged with WA1/2020 (left) or Omicron BA.1 (n=5) (right). Viral RNA in the lungs was quantified 3-dpi. (D) Mice were administered luciferase-expressing lentiviral vector i.v. or i.n. (n=2). Tissues (nasal, lung, trachea, spleen, and liver) were harvested and luciferase activity was measured over 60 days at the indicated time-points. (E) Decoy-expressing lentiviral vectors (5×10⁶ IU) were administered i.v. or i.n. and after 7-, 30- and 60-days challenged with SARS-COV-2 (1×10⁴ PFU). Viral RNA was quantified 3-dpi. Confidence intervals are shown as the mean±SD. ** P≤0.01, *** P≤0.001, **** P≤0.0001. The experiment was done twice with similar results.

FIG. 5. Comparison of lung cell subpopulations transduced by AAV and lentiviral vectors. (A) GFP-expressing AAV2.retro and AAV6.2 were administrated i.n. After 3 days, the lungs were harvested and the tissue enzymatically disaggregated. The cells were analyzed by multi-color flow cytometry with cell-type specific marker antibodies to distinguish subpopulations defined as follows: Leukocytes (CD45+), epithelial (CD45−), alveolar macrophages (CD45+, F4/80+, SiglecF+), interstitial macrophages (CD45+, F4/80+, SiglecF−), DCs (CD45+, F4/80−, CD11c+), T cells (CD45+, CD3+), B cells (CD45+, CD19+), monocytes (CD45+, CD11b+, CD14+), neutrophils (CD45+, CD62L+, Ly6C/Ly6G+). Representative flow cytometry plots of the GFP+ cells and GFP+/CD45+ populations are shown on the top and the subpopulations within the GFP+/CD45+ leukocytes are shown in the pie charts on the bottom. (B) Mice were administered GFP-expressing lentiviral vector i.n. GFP+ cells in the lung were analyzed by flow cytometry as in (A). Representative flow cytometry plots of the GFP+ cells and GFP+/CD45+ populations are shown on the left and the subpopulations within the GFP+/CD45+ leukocytes are shown in the pie charts in (A).

FIG. 6. Neutralization of new omicron variants by decoy. (A) Serially diluted ACE2.1mb was incubated with Omicron pseudotypes. Infectivity was measured on ACE2.293T cells by NLuc assay. Calculated IC50s are shown in the table. (B) Catalytic activity of ACE2mb (WT), ACE2mb (H345A) and ACE2mb (R273A+H345A) proteins was measured by assay with Mca-APK(Dnp) or Ang-II. ACE2 activity was measured by fluorescence at 405 nm. Ang-Il cleavage by decoys was measured by phenylalanine assay. (C) A fixed amount of WT ACE2mb (1 μg) was mixed with increasing amounts of ACE2mb (R273A++H345A) in the activity assay. The rate of Ang-Il cleavage was measured in the phenylalanine assay.

FIG. 7. Decoys do not induce anti-drug antibody (ADA). (A) C57BL/6 mice (n=3) were injected with 100 μg of ACE2.1mb i.n. or i.v. 1- and 2-week post-decoy administration, serum and bronchoalveolar lavage (BAL) were obtained. (B) Decoy concentration in serum and BAL were measured by neutralization of D614G spike lentiviral pseudotype. Decoy concentration (μg/ml) is shown in the table. (C) ADA in serum and BAL were measured by ELISA. Tissue samples were added to decoy-coated microtiter plate. Decoy-bound antibody was detected with goat-HRP-anti-mouse IgG with HRP substrate. Serum from a mouse immunized with decoy protein was used as a positive control.

FIG. 8. Decoy does not induce inflammatory cytokines. Mice (n=4) were injected with 100 μg of ACE2.1mb i.n. or i.v. 1- and 2-weeks post-decoy administration, IFNγ, TNFα, IL-10, MCP-1 and IL-12p70 were measured in the serum (top) and BAL (bottom) by cytokine beads array. Serum and BAL from SARS-COV-2 WA/2020 infected mice were used as a positive control. Cytokines are indicated on the Y-axis.

FIG. 9. Intramuscular injection of decoy-expressing AAV2.retro protects long-term. (A) hACE2-Tg were treated i.m., i.v., or i.n. with decoy-expressing AAV2.retro (1×10¹²vg). 1-week, 2-weeks, 4 weeks, and 3-months post-AAV injection, the mice were challenged with SARS-COV-2 WA1/2020. (B) Viral RNA was quantified 3-dpi. (C) Decoy concentration in the serum was quantified by lentivirus inhibition assay. UD: Undetected.

FIG. 10. AAV-decoy does not induce ADA or inflammatory cytokine secretion. (A) ELISA measurement of serum antibody against decoy in ACE2 transgenic or C57BL/6 mice treated i.m., i.n., or i.v. with decoy-expressing AAV2.retro. (B) Mice (n=3) were administered decoy-AAV2.retro (1×10¹²vg) i.m., i.n., or i.v. 1- and 2-weeks, 1- and 3-months post-decoy administration, proinflammatory cytokines IFNγ, TNFα, IL-10, IL-6, MCP-1 and IL-12p70 in the serum were measured by cytokine bead array. Serum of SARS-COV-2 WA/2020-infected mice was used as a positive control. Cytokines are indicated on the Y-axis.

FIG. 11. AAV-Decoy protected mice from SARS-COV-2 infection. Different dose of AAV2.retro or AAV6.2 decoy (1×10¹², 1×10¹¹, 1×10¹⁰, 1×10⁹ vector genomes) were administered to hACE2 K18 Tg (n=4) by i.n. instillation. Three days post-AAV injection, mice were challenged with 1×10⁴ PFU of SARS-COV-2 WA1/2020. At 3-dpi, subgenomic viral E gene RNA in the lung were quantified.

FIG. 12. Gating strategies to identify GFP+ cells in different cell types in mouse lung. C57/B6 mice were injected with AAV-GFPNLuc virus. Three days post injection, the GFP+ cells in the lung were analyzed. Each of the cell types was classified as follows: epithelia (CD45−), alveolar macrophage (CD45+, F4/80+, SiglecF+), interstitial macrophages (CD45+, F4/80+, SiglecF−), DC (CD45+, F4/80−, CD11c+), T cells (CD45+, CD3+), B cells (CD45+, CD19+), monocytes (CD45+, CD11b+, CD14+), neutrophils (CD45+, CD62L+, Ly6C/Ly6G+).

FIG. 13 Lentivirus-based ACE2.1mb was transduced in CHME3.ACE2 and A549.ACE2 cells. CHME3.ACE2 and A549.ACE2 cells were transduced with pLenti-ACE2.1mb at an MOI of 0.5. Three days post-infection, decoy protein secreted into the supernatant was pulled-down on NTA beads and bead-bound decoy protein was detected on an immunoblot probed with His-tag antibody. Decoy protein in the cell lysates is shown below with GAPDH as a loading control.

FIG. 14. Lentivirus-based ACE2.1mb protects mice from SARS-COV-2 infection. hACE2-Tg mice were injected i.v. or i.n. with different amounts of pLenti.ACE2.1mb. One week later, the mice were challenged with 1×10⁴ PFU SARS-COV-2 (n=3). AT 3-dpi, lung subgenomic viral E RNA was quantified by RT-PCR. Confidence intervals are shown as the mean ±SD. ** P≤0.01, *** P≤0.001, **** P≤0.0001.

FIG. 15. AAV and lentivirus-based ACE2.1mb protects mice from the SARS-COV-2 induced cytokine storm. Mice were treated with the decoy-expressing AAV (1×10¹² vg) and lentiviral vector (5×10⁶ IU). After seven days, the mice were challenged with WA12020 and 3-dpi the levels of IFNg, TNFα, IL-6 IL-10 IL-12 and MCP-1 in lung were measured by cytokine beads array. The Y-axis shows the concentration of each cytokine. The experiment was done twice with similar results.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

Any database entry reference, such as reference to a sequence database, incorporated herein the sequence associated with the database entry as it exists on the effective filing date of this application or patent.

The disclosure includes all polynucleotide and amino acid sequences described herein expressly and by reference, and every polynucleotide sequence referred to herein includes its complementary sequence, and its reverse complement. All segments of polynucleotides from 10 nucleotides to the entire length of the polynucleotides, inclusive, and including numbers and ranges of numbers there between are included. DNA sequences includes the RNA equivalents thereof to the extent an RNA sequence is not given. Every DNA and RNA sequence encoding polypeptides disclosed herein is encompassed by this disclosure, including but not limited to sequences encoding all recombinant proteins that comprise any complete protein or segment thereof. Every amino acid sequence disclosed herein includes every polynucleotide sequence encoding it. All of the amino acid sequences and nucleotide sequences associated with any database accession numbers are incorporated herein by reference as they exist in the database as of the date of the filing of this application or patent. The disclosure includes all polynucleotide and protein sequences described herein expressly or by reference that are between 80.0% and 99.9% identical to the described sequences. The proteins may comprise one or more than one amino acid changes, in addition to the amino acid change described herein which inhibits the enzymatic function of the ACE2 protein. Such changes can comprise conservative or non-conservative amino acid substitutions, insertions, and deletions, and the like. Any one or combination of components can be omitted from the claims, including any polynucleotide sequence, any amino acid sequence, and any one or combination of steps. Where reference to a fusion protein is made in this disclosure, expression vectors encoding the fusion protein are included in the disclosure. Any amino acid sequence described herein, and any polynucleotide encoding any amino acid sequence, can omit a segment of amino acids, such as those used for purification, including but not limited to a series of His residues at the C-terminus of the protein comprising such amino acid sequence. The disclosures of U.S. provisional patent application No. 63/067,304, filed Aug. 18, 2020, and U.S. patent application Ser. No. 17/405,104, filed Aug. 18, 2021, published as U.S. patent publication no. 20220056429-A1, are incorporated herein by reference.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” or “approximately” it will be understood that the particular value forms another embodiment. The term “about” and “approximately” in relation to a numerical value encompasses variations of +/−10%, +/−5%, or +/−1%.

The present disclosure provides improved compositions and methods for combatting COVID-19, and other Coronaviruses that express an S protein that binds to ACE2, such as any β-Coronavirus. The compositions comprise in certain embodiments an expression vector encoding an ACE2-“microbody” in which the ACE2 ectodomain is fused to an lgG CH3 domain of an IgG Fc region. The Examples below demonstrate development of lung cell-tropic AAV vectors expressing a high affinity ACE2 microbody and their effectiveness in mouse models. The Examples show that treatment of uninfected mice provided long-term protection from high dose infection with SARS-COV-2 and that treatment of SARS-COV-2-infected mice resulted in a rapid drop in virus loads when given shortly post-infection. To extend the length of decoy expression, the disclosure also includes a lentivirus-based decoy. The lentivirus-based decoy, administered either by i.v. injection or i.n. instillation, profoundly suppressed virus replication upon subsequent challenge with live virus. The immunity was durable, showing no sign of diminishing after two months and most likely longer, suggesting that long-lived cells residing in the lung and spleen had been transduced. In mice injected i.v. with a GFP-expressing lentiviral vector, GFP+ cells were localized to the spleen where their numbers remained constant over 2 months (Tada et al., 2022), supporting long-lasting immunity provided by the vector. While i.n. instillation resulted in the production of the decoy in the respiratory tract, the primary site of SARS-COV-2 infection, i.v. injection, which primarily results in the transduction of splenocytes (Tada et al., 2022) was also effective. The findings suggest that the decoy protein produced by splenocytes is secreted into circulation and is then distally distributed, maintaining a local concentration sufficient to suppress virus replication. The disclosure therefore demonstrates in some embodiments that systemic distribution of the protein is expected to be advantageous for the suppression of virus replication in sites such as the lung as well as other tissues of the body. In embodiments the described approach is expected to be useful for the protection of immunocompromised individuals for whom vaccination may be less effective. Therapeutic approaches for use in infected individuals are also provided, including for vaccinated and unvaccinated individuals.

As discussed above, the disclosure includes but is not necessarily limited to expression vectors and uses thereof, wherein the expression vectors encode a described ACE2-microbody in which the ACE2 ectodomain is fused to an IgG CH3 domain of an lgG Fc region. In embodiments, the ACE2 ectodomain may be enzymatically inactive, or its enzymatic activity may be reduced by modification of one or more amino acids such that the ACE2 component is less enzymatically active than an unmodified version. In embodiments, the ACE2 component of a described fusion protein does not comprise an intact ACE2 transmembrane domain, an intact cytoplasmic tail, or does not comprise a combination thereof.

The amino acid sequences of IgG CH3 domains of lgG Fc segments are known in the art. In embodiments, a fusion protein of the disclosure comprises an approximately or precisely 740 amino acid sequence segment of the ACE2 protein. In embodiments one or more amino acid sequences in the ACE2 protein may be changed. In the ACE2 human protein, the extracellular domain is amino acids 1-740, the transmembrane (TM) domain is amino acids 741-763, and the cytoplasmic tail is amino acids 764-805. The full-length human ACE2 protein sequence is:

(SEQ ID NO: 1)
MSSSSWLLLSLVAVTAAQSTIEEQAKTFLDKFNHEAEDLFYQSSL
ASWNYNTNITEENVQNMNNAGDKWSAFLKEQSTLAQMYPLQEIQN
LTVKLQLQALQQNGSSVLSEDKSKRLNTILNTMSTIYSTGKVCNP
DNPQECLLLEPGLNEIMANSLDYNERLWAWESWRSEVGKQLRPLY
EEYVVLKNEMARANHYEDYGDYWRGDYEVNGVDGYDYSRGQLIED
VEHTFEEIKPLYEHLHAYVRAKLMNAYPSYISPIGCLPAHLLGDM
WGRFWTNLYSLTVPFGQKPNIDVTDAMVDQAWDAQRIFKEAEKFF
VSVGLPNMTQGFWENSMLTDPGNVQKAVCHPTAWDLGKGDFRILM
CTKVTMDDFLTAHHEMGHIQYDMAYAAQPFLLRNGANEGFHEAVG
EIMSLSAATPKHLKSIGLLSPDFQEDNETEINFLLKQALTIVGTL
PFTYMLEKWRWMVFKGEIPKDQWMKKWWEMKREIVGVVEPVPHDE
TYCDPASLFHVSNDYSFIRYYTRTLYQFQFQEALCQAAKHEGPLH
KCDISNSTEAGQKLFNMLRLGKSEPWTLALENVVGAKNMNVRPLL
NYFEPLFTWLKDQNKNSFVGWSTDWSPYADQSIKVRISLKSALGD
RAYEWNDNEMYLFRSSVAYAMRQYFLKVKNQMILFGEEDVRVANL
KPRISFNFFVTAPKNVSDIIPRTEVEKAIRMSRSRINDAFRLNDN
SLEFLGIQPTLGPPNQPPVSIWLIVFGVVMGVIVVGIVILIFTGI
RDRKKKNKARSGENPYASIDISKGENNPGFQNTDDVQTSF

The disclosure includes all contiguous segments of SEQ ID NO:1 that are at least 10 contiguous amino acids in SEQ ID NO:1. In these and other embodiments, position 345 in the above described SEQ ID NO:1 is not a His, and may substituted with, for example, an Ala, or other suitable amino acid such that the protease activity of the protein is reduced or eliminated. In an embodiment, position 273 in SEQ ID NO:1 is not an R, and may be substituted with, for example, an Ala such that the protease activity of the protein eliminated. Representative and non-limiting examples of polynucleotide and amino acid sequences included in the disclosure are provided in the Tables that follow the Examples.

In embodiments, the microbody segment of the fusion protein is approximately or precisely 131 amino acids of the above described SEQ ID NO:1. Without intending to be constrained by any particular theory it is considered the microbody facilitates dimerization of fusion proteins, and provides additional advantages for therapeutic approaches, such as an improved half-life, and inhibits or prevents binding of the fusion protein to Fc receptors.

In embodiments, a fusion protein of the disclosure comprises approximately or precisely 879 amino acids, which comprises or consists of a described ACE2 protein segment, the microbody segment, and a mutation at position 345. In embodiments, the fusion protein is a contiguous polypeptide that does not include a linker sequence between the soluble, mutated ACE2 and microbody segments, but a linker may be used if desired.

In embodiments, a viral expression vector may be used for introducing one or more of the components of the described fusion protein system. Viral expression vectors may be used as naked polynucleotides, or may comprises viral particles. In embodiments, the expression vector comprises a modified viral polynucleotide, such as from an adenovirus, a herpesvirus, or a retrovirus, such as a lentiviral vector. Representative and non-limiting examples of suitable lentiviral vectors are discussed herein. In embodiments, one or more components of the described of system may be delivered to cells using, for example, a recombinant adeno-associated virus (AAV) vector. Representative and non-limiting examples of suitable AAV vectors are discussed herein. AAV vectors are also commercially available, such as from TAKARA BIO® and other commercial vendors, and may be adapted for use with the described systems, given the benefit of the present disclosure. In certain embodiments, the expression vector is a self-complementary adeno-associated virus (scAAV). In an embodiment, mRNA encoding a described fusion protein may be introduced into an individual in need thereof.

In embodiments, a composition of the disclosure is administered to an individual who is infected with SARS-COV-2, or is suspected of having a SARS-COV-2 infection, or another Coronavirus that causes a deleterious infection.

In embodiments, the composition is administered to an individual who is at risk for contracting a Coronavirus infection, including but not necessarily limited to a SARS-CoV-2 infection. In embodiments, the individual is a human and is of an age wherein such risk is heightened, such as any individual over the age of 50 years. In embodiments, the individual has an underlying condition wherein the risk of developing severe symptoms of a Coronavirus infection, such as COVID-19, is increased, including but not necessarily limited to any respiratory condition. In embodiments, the individual is not eligible to receive a SARS-CoV-2 vaccine. In embodiments, the individual has a condition that results in a compromised immune system. In embodiments, the disclosure includes veterinary approaches, such as for administration to domesticated felines who have or are at risk of developing Feline infectious peritonitis (FIP).

In embodiments, the individual has been diagnosed with or is at risk of contracting a SARS-COV-2 variant infection. Such variants include but are not necessarily limited to variants currently referred to as variants of interest, variants of concern, and variants of high consequence. In embodiments, the described polypeptides can be administered to an individual who has been diagnosed with or is at risk for contracting a SARS-COV-2 infection wherein the virus comprises any mutation described herein, such as a SARS-COV-2 spike mutation.

In embodiments, an effective amount of a composition is administered to an individual. An effective amount means an amount of the fusion protein or a vector encoding the fusion protein, whereby production of the fusion protein will elicit the biological or medical response by a subject that is being sought by a medical doctor or other clinician. In embodiments, an effective amount means an amount sufficient to prevent, or reduce by at least about 30 percent, or by at least 50 percent, or by at least 90 percent, any sign or symptom of viral infection, e.g., any sign or symptom of COVID-19. In embodiments, fever is prevented or is less severe than if the presently described vaccine had not been administered. In embodiments, viral pneumonia is inhibited or prevented. In embodiments, binding of a Coronavirus to an ACE2 receptor, e.g., binding of a Coronavirus spike protein to an ACE2 receptor, is inhibited or prevented. Thus, in embodiments, viral entry into cells that express the ACE2 inhibitor, including but not necessarily limited to respiratory epithelial cells, can be inhibited or prevented. In embodiments, use of the described polypeptides reduce SARS-COV-2 viral genome copy number, and/or reduce SARS-COV-2 virus entry into human cells, and/or inhibit cell death caused by SARS-COV-2 infection.

In embodiments, the described polypeptides or a vector encoding the polypeptides may be provided as pharmaceutical formulations. A pharmaceutical formulation can be prepared by mixing the polypeptides with any suitable pharmaceutical additive, buffer, and the like. Examples of pharmaceutically acceptable carriers, excipients and stabilizers can be found, for example, in Remington: The Science and Practice of Pharmacy (2005) 21st Edition, Philadelphia, PA. Lippincott Williams & Wilkins, the disclosure of which is incorporated herein by reference.

In embodiments, a described polypeptide or vector encoding the polypeptide may be administered with another agent, including but not necessarily limited to anti-viral and anti-inflammatory agents. The polypeptide and additional agent(s) may be administered concurrently or consecutively. The described polypeptide whether administered as such or by using a vector encoding it may potentiate the effect of another anti-viral agent. In embodiments, a combination of a described agent and one or more additional agents may exhibit a synergistic anti-viral effect. In embodiments, a described polypeptide or vector encoding it may be administered to an individual with one more iminosugars, and/or with one or more antiviral compounds, non-limiting example of which includes nucleoside analogs such as Remdesivir and Galidesivir. In embodiments, a combination of a described polypeptide and an anti-viral antibody is administered. In a non-limiting approach, the polypeptide or vector encoding it and one or more anti-SARS-COV-2 antibodies are administered to an individual in need thereof. In an embodiment, at least one of the anti-SARS-COV-2 antibodies bind with specificity to an epitope on the viral spike protein. In embodiments, the antibodies are one or both of antibodies known in the art as such as REGN10933 and REGN10987 or other current or future therapeutic anti-spike protein monoclonal antibodies. In embodiments, a combination of the described polypeptide or a vector encoding it and one or more anti-SARS-COV-2 antibodies produce synergistic viral neutralization. In embodiments, a combination of agents described herein maintains its ability to inhibit current spike protein variants and is predicted to inhibit future variants that may emerge, so long as they maintain use of ACE-2 as receptor.

Methods of making the described fusion proteins are also included, such as by expressing the fusion protein in or a vector encoding the fusion protein in a plurality of cells, and separated the express fusion protein or viral particles modified to encode the described fusion proteins from the cells.

Administration of formulations comprising the fusion proteins of this disclosure can be performed using any suitable route of administration, including but not limited to parenteral, intraperitoneal, and oral administration. Parenteral infusions include intramuscular, intravenous, intraarterial, intraperitoneal, and subcutaneous administration. In an embodiment, a described fusion protein or vector encoding it is administered orally, or by inhalation, including but not necessarily limited to nasal administration. The compositions can be administered to humans, and are also suitable for use in a veterinary context and accordingly can be given to non-human animals, including non-human mammals. In embodiments, a single administration is administered and is sufficient for a therapeutic response. In embodiments, more than one administration is provided.

The following Examples are intended to illustrate but not limit the disclosure.

### Example 1

### Decoy-Expressing AAV Vectors Inhibit SARS-COV-2 Infection.

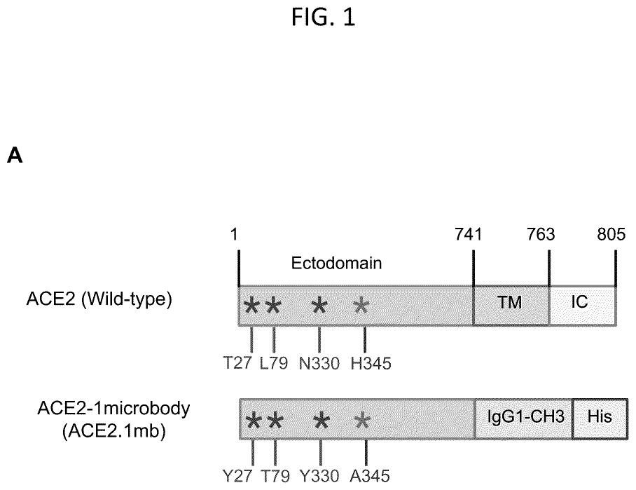

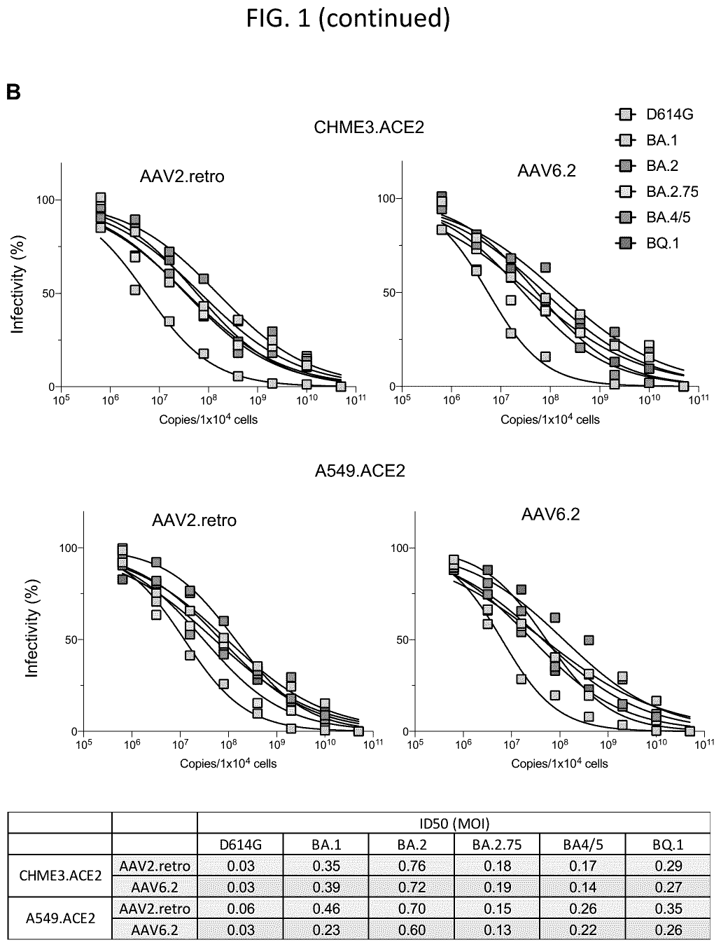

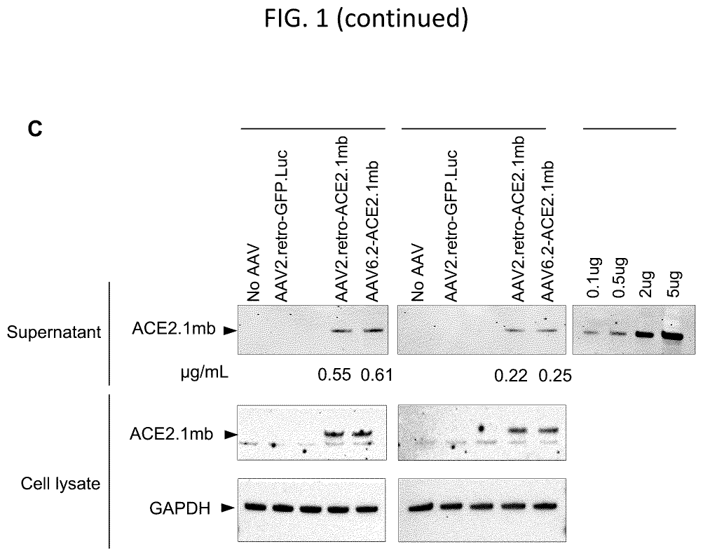

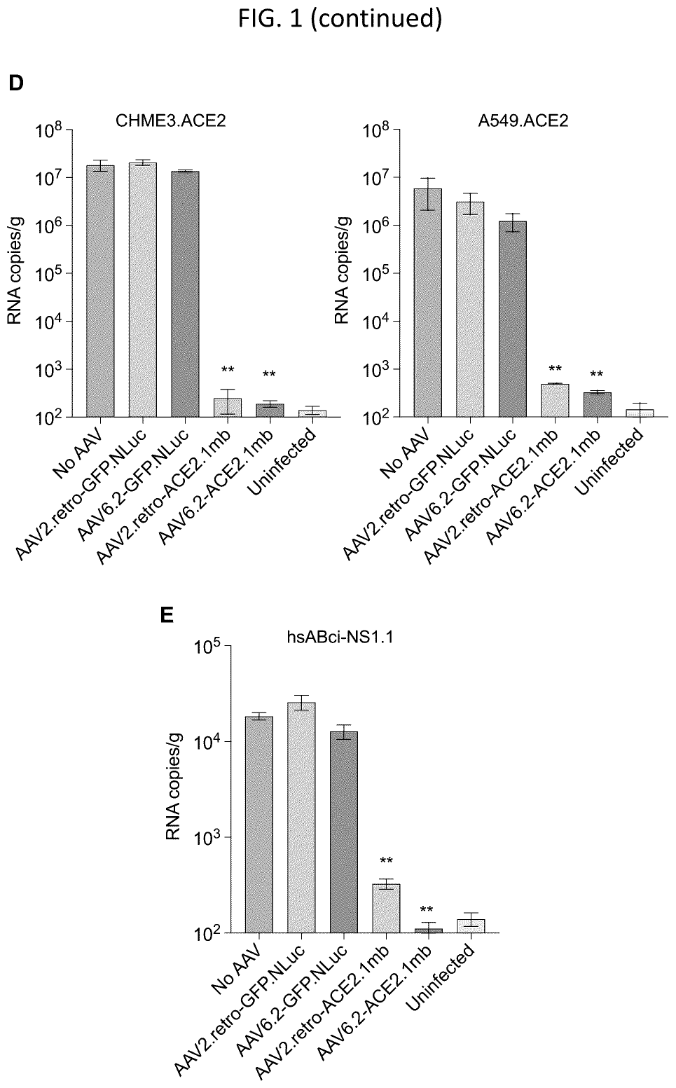

To determine the feasibility of vectored prophylaxis for SARS-COV-2, we constructed AAV vectors expressing an ACE2 receptor decoy. The decoy comprises three point mutations to increase ACE2 affinity. The decoy, termed ACE-2.mb.2.0, comprises the ACE2 ectodomain fused to a single CH3 domain of an immunoglobulin Fc region (FIG. 1A). The decoy gene was cloned into CMV- and EF2a-promoter AAV vectors and virus stocks were produced with AAV2.retro, AAV6 and AAV6.2 capsids. AAV2 and AAV6 have reported tropism for mouse and human airway cells. AAV2.retro is a variant of AAV2 that was selected for increased tropism for the central nervous system (CNS) and retrograde movement in axons (Tervo et al., 2016; Zhang et al., 2022). AAV6.2 is a variant of AAV6 containing a single F129L mutation that was found to increase the transduction efficiency of mouse and human airway cells (Limberis et al., 2009). The ability of the vectors to protect cells from SARS-COV-2 infection was tested in the lung cell-line A549.ACE2 and microglial cell-line CHME3.ACE2. The cells were transduced with serially diluted decoy vector and then challenged with lentiviral pseudotyped by the spike proteins of D614G, BA.1, BA.2, BA.2.75, BA.4/5 and BQ.1. After two days, luciferase activity in the cultures was measured. The results showed that the AAV2.retro and AAV6.2 decoy vectors neutralized the parental D614G spike virus and the Omicron variants in the A549.ACE-2 and CHME3.ACE-2 cells (FIG. 1B). Virus with the D614G spike was the most potently neutralized while BA.2 was the most resistant, with a ID50 about 10-fold higher. Production of the decoy protein in the transduced cells was confirmed by pull-down of the protein with anti-His tag coated magnetic beads (FIG. 1C). The results showed that the CHME3.ACE2 cells produced about 2-fold more of the decoy, contributing to the greater extent of protection in these cells. The concentration of the decoy in the culture medium was about 0.5 g/ml which was great than the IC50 0.15 μg/ml of decoy protein.

The ability of the decoy-expressing vectors to inhibit SARS-COV-2 live virus replication was tested on A549.ACE2, CHME3.ACE2 and, in addition, on human lung hSABCi-NS1.1 cell that had been grown in air-liquid interface culture conditions and differentiated into mature airway epithelium. All three cell-lines support high levels of SARS-CoV-2 replication. The cells were transduced with AAV2.retro and AAV6 decoy-expressing vectors or control GFP.nLuc vector and challenged a day later with SARS-COV-2 WA1/2020. Virus replication was measured by RT-qPCR quantification of cell-associated viral RNA copies. The results showed high levels of virus replication in A549.ACE2 cells transduced with the control vectors and a 3-4 log decrease in SARS-COV-2 RNA upon transduction with the decoy-expressing AAV2 and AAV6 vectors, a decrease that approached undetectable levels (FIG. 1D). The results in CHME3.ACE2 cells were even more pronounced with a 4-5 log decrease in viral RNA (FIG. 1D). The decrease was less pronounced in the airway epithelium cultures of hSABCi-NS1.1 cell (50-100-fold decrease in viral RNA) that most likely resulted from decreased SARS-COV-2 replication in these cells (FIG. 1E).

### Example 2

### Vectored Prophylaxis In Vivo by Decoy-Expressing AAV-Vectors

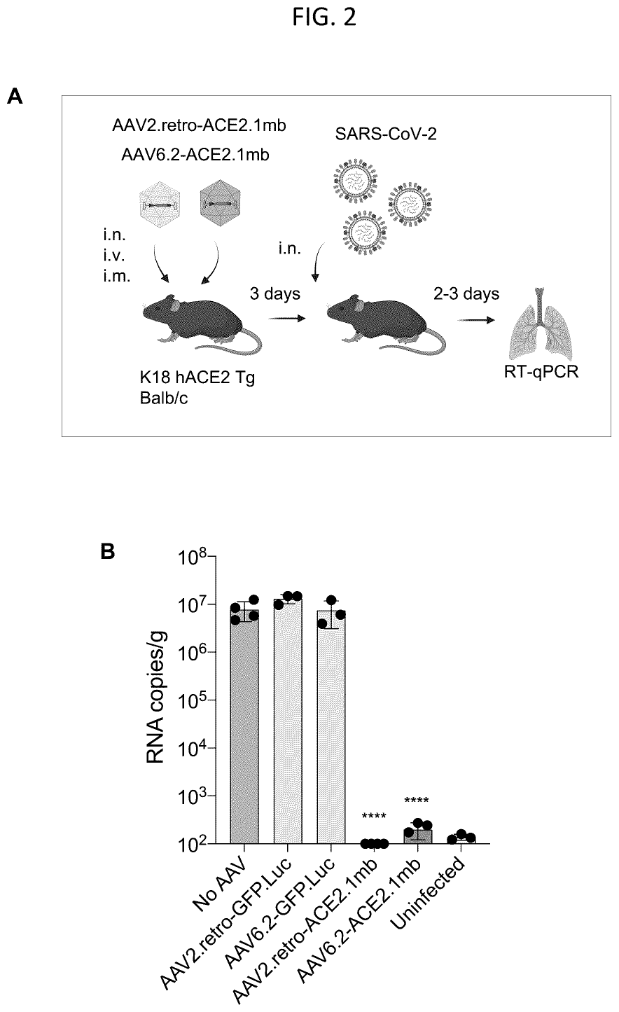

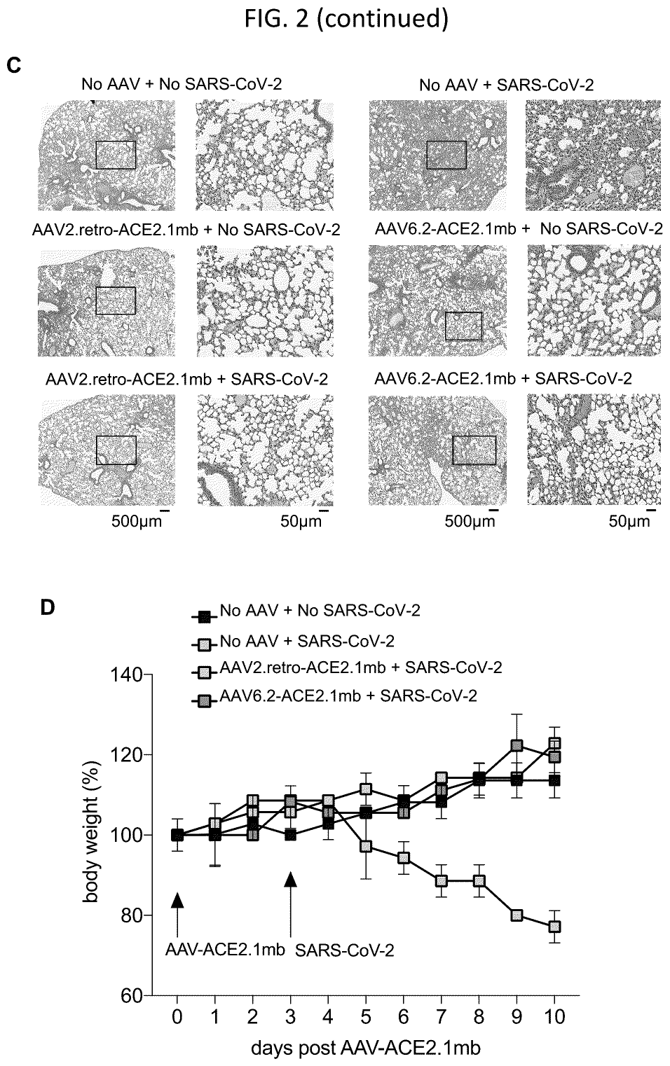

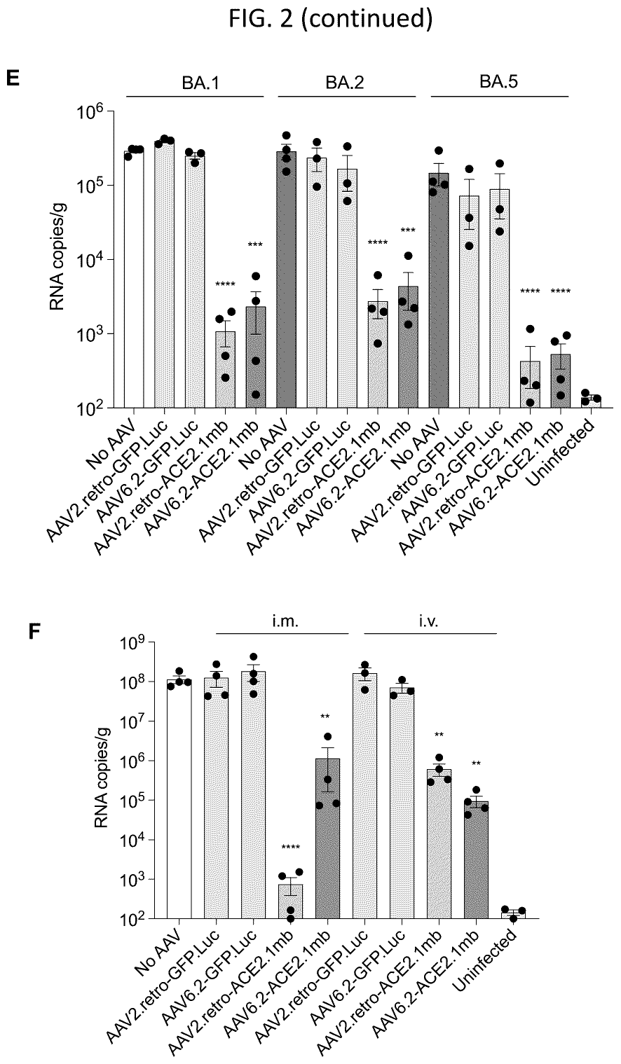

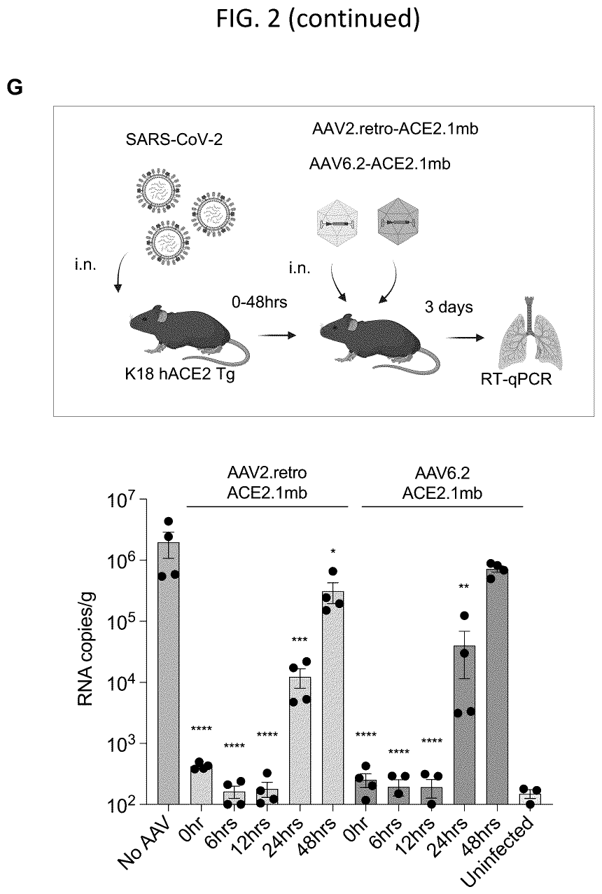

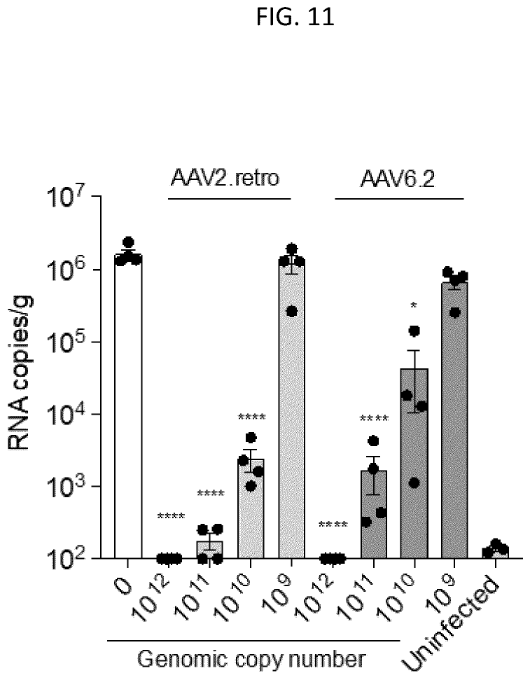

The feasibility of vectored prophylaxis for SARS-COV-2 was tested in mouse models. The decoy-expressing AAV2.retro and AAV6.2 vectors were administered i.v. and i.n. to ACE2 transgenic K18 mice (hACE2 K18 Tg) and after 3 days, the mice were challenged with SARS-COV-2 WA1/2020 (FIG. 2A). Virus loads in the lung were measured 3 days post-infection. The results showed that i.n. administration of the vectors strongly suppressed virus replication in the mice, decreasing the virus load by 5-logs, a level indistinguishable from uninfected mice (FIG. 2B). Histology showed that the lungs of untreated infected mice had prominent signs of interstitial pneumonia with thickened alveolar septa and inflammatory cell infiltration (FIG. 2C) while the lungs of decoy-expressing AAV vector-treated mice showed no signs of pneumonia and were free of infiltrating inflammatory cells. Treatment with the decoy vectors prevented the characteristic loss of body mass associated with untreated SARS-COV-2 infection (FIG. 2D). In addition, the lungs of mice treated with the decoy AAV vectors alone in the absence of SARS-COV-2 infection were clear, indicating that the decoy vectors themselves did not cause pulmonary inflammation (FIG. 2C). The effectiveness of the decoy vector treatment in protection against the Omicron variants was tested in Balb/c mice which support high levels of replication of the variant using the endogenous murine ACE2. Administration of the decoy-expressing AAV2.retro and AAV6.2 vectors by the i.n. route suppressed the replication of Omicrons BA.1, BA.2 and BA.5 as compared to mice treated with control AAV2.retro.GFP and AAV6.2 (FIG. 2E). The vectors were most effective against the BA.5 variant, decreasing virus load 1000-fold and least effective against BA.2, decreasing virus load 100-fold. The vectors were effective when administered i.m., but less effective when administered i.v.; AAV2.retro and AAV6.2 decoy vectors decreased the virus load 20- and 100-fold, respectively (FIG. 2F). A dose-response analysis showed that the decoy-expressing AAV2.retro vector was the more potent of the two vectors. It maintained its ability to suppress virus loads when used at a 10-fold-decrase in dose; at a 100-fold decrease in dose, the vector still decreased virus the load 200-fold. The AAV6.2 vector was about 20-fold less potent, decreasing the virus load 1000-fold when diluted 10-fold (1×10¹¹ vg) (FIG. 11).

### Example 3

### Decoy-Expressing AAV Vectors are Also Effective Therapeutically.

The experiments described above tested the use of the decoy-expressing vectors for SARS-COV-2 prophylaxis. Whether the vectors could be used therapeutically would depend on how soon post-infection they were administered and how rapidly the vectors transduce lung cells and establish a sufficient concentration of decoy protein in the respiratory tract. To determine this, we infected mice with SARS-COV-2 and then treated at increasing times post-infection with the decoy-expressing AAV2.retro and AAV6.2 vectors (FIG. 2G). Viral RNA copy numbers in the lung were measured 3 days post-infection. The results showed that the decoy-expressing vectors were highly effective when implemented concomitant with SARS-COV-2 infection and up to 12 hours post-infection (FIG. 2G). The treatment was partially effective at 24 hours post-infection and lost efficacy at 48 hours. The results show the that the vector treatment is effective highly effective when provided soon after infection. This kinetics closely mirror what is seen for monoclonal antibody therapy for SARS-COV-2 in mouse models.

### Example 4

### AAV Decoy-Expressing Vectored Prophylaxis is Highly Durable.

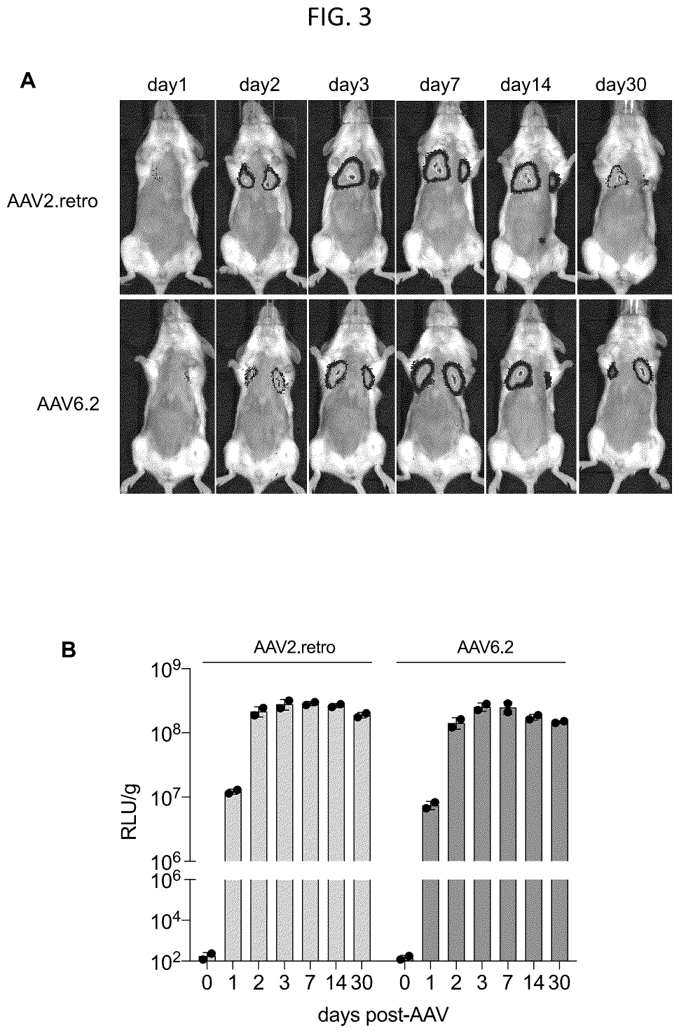

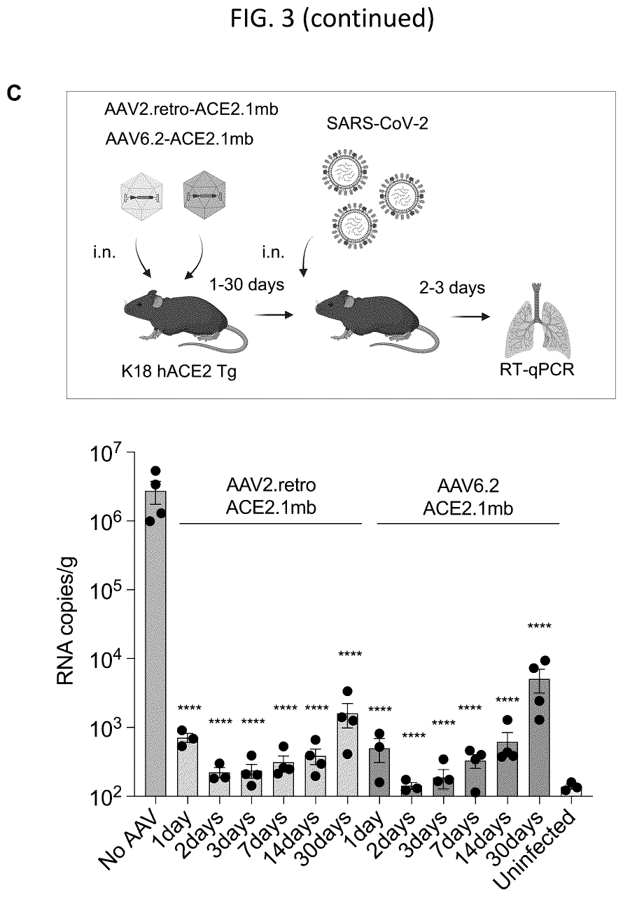

To test the time-course of vector expression, AAV2.retro and AAV6.2 vectors were constructed that expressed a decoy-luciferase fusion protein. The vectors were administered i.n. and the mice were live-imaged over 30 days. Expression from both vectors in the lung was first detected 24 hours post-treatment and then increased to maximal by day 3 (FIG. 3A); the expression level remained stable through day 14 after which it decreased slightly on day 30. Measurement of luciferase activity in tissue homogenates further demonstrated the long-lived expression by the vectors (FIG. 3B). The analysis confirmed the slight decrease in expression level at 30 days, coinciding with the small decrease in viral load suppression found in the prophylaxis experiments described above (FIGS. 3A and 3B). The AAV2.retro vector appeared to express slightly better than the AAV6.2 vector although this difference did not reach statistical significance (FIG. 3B). The decoy proteins were readily detectable in the homogenates on day 1 at a level about 25-fold less than on day 2 (FIG. 3B).

We next examined the durability of protection established by the decoy-expressing AAV vectors was tested in a time-course analysis in which mice were treated with the vectors and then challenged over a 30-day period with SARS-COV-2 WA1/2020. In this analysis, the AAV2.retro and AAV6.2 vectors both maintained potent virus load suppression over the 30-day time course (FIG. 3C). At 30 days, there was a small increase in virus loads that was more apparent in the AAV6.2 vector.

### Example 5

### Lentiviral Vectored Prophylaxis.

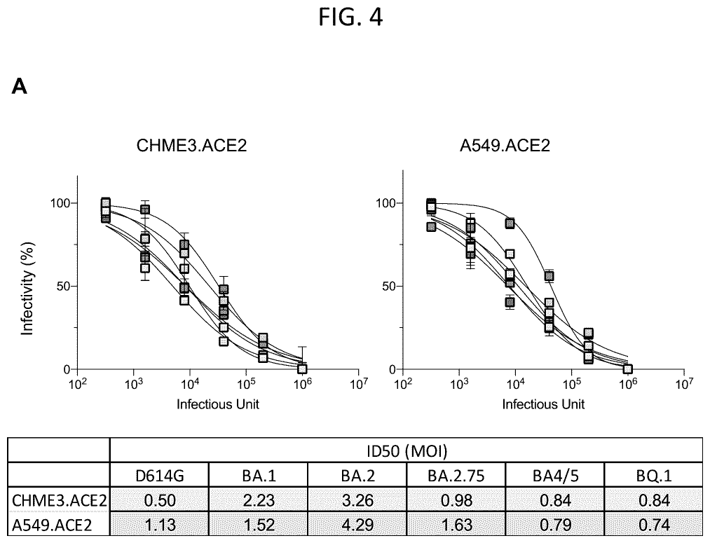

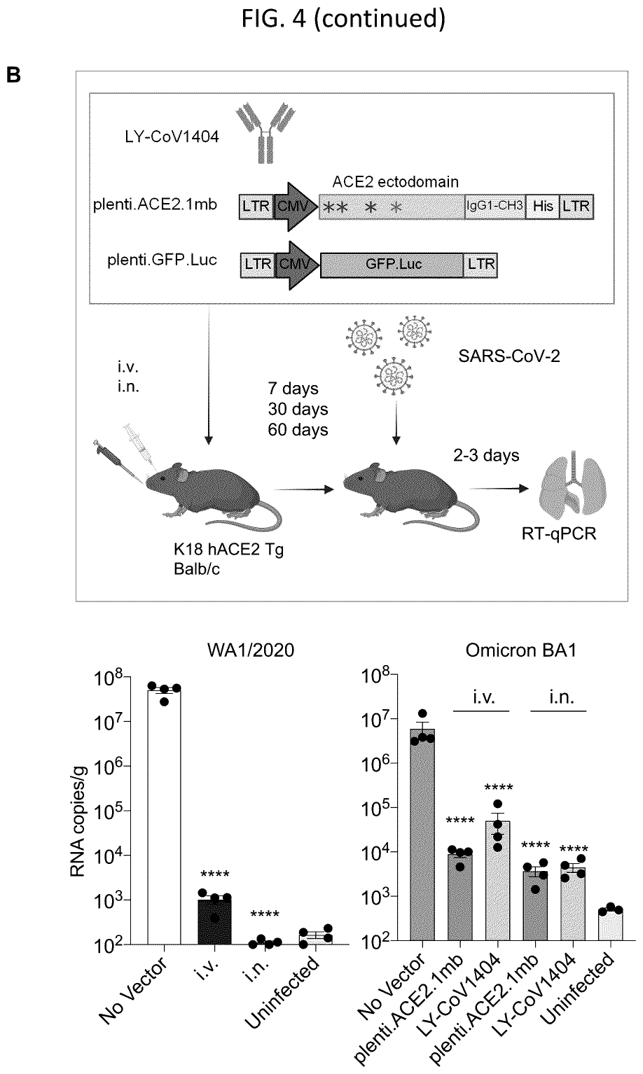

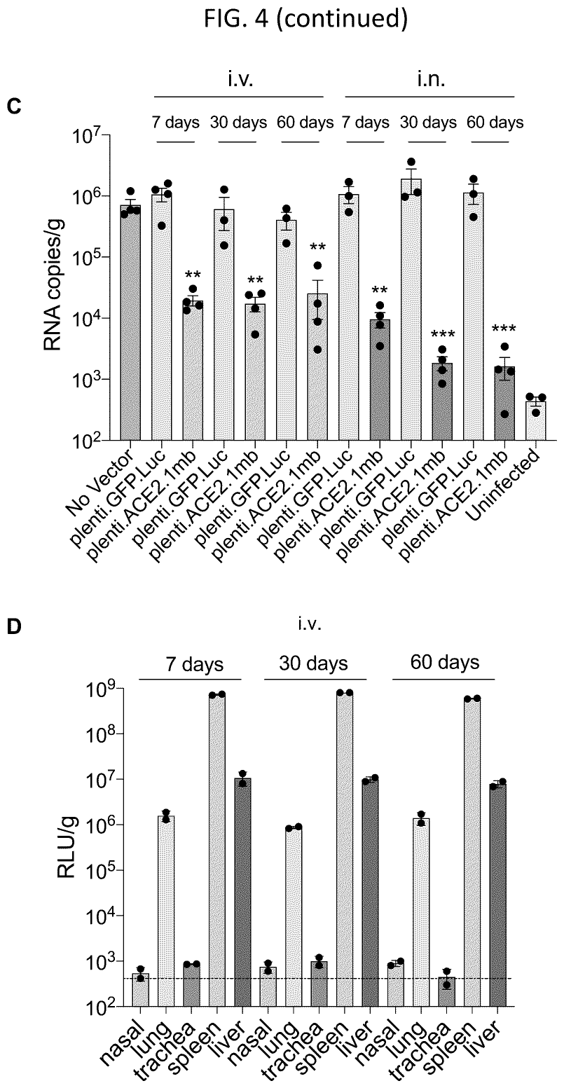

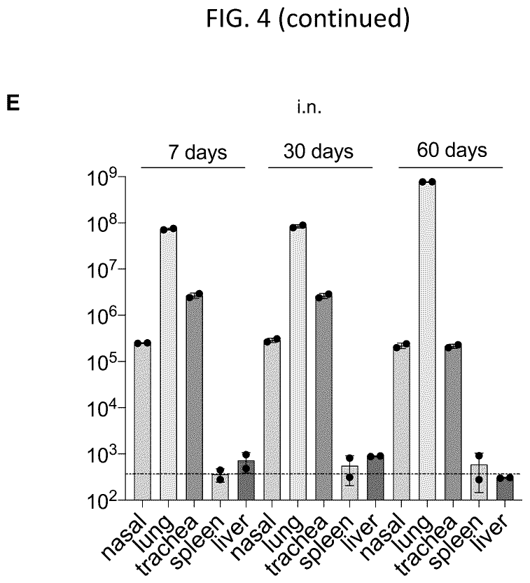

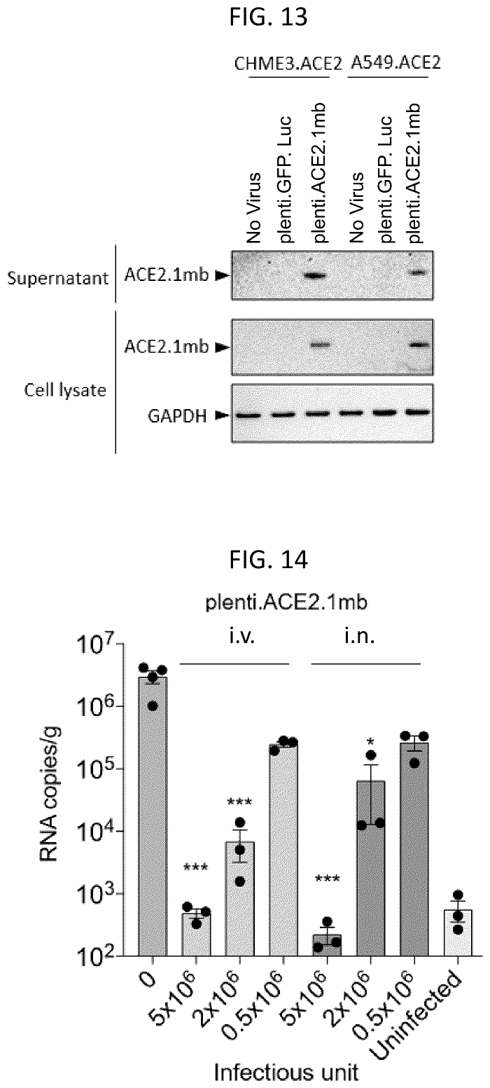

Pre-existing immunity to AAV in some individuals can be a concern in gene therapy applications for these vectors. In addition, the small decrease in protection at 30 days post-treatment, suggested that it would be of interest to test whether even longer-term protection could be established using an alternative vector. Lentiviral vectors, by virtue of their ability to integrate into the host cell genome, potentially allow for life-long expression in the host cell and daughter cells that may be generated over time and are generally not subject to pre-existing immunity. Moreover, they have a broad target cell tropism by virtue of the VSV-G pseudotyping. To test the feasibility of lentiviral vectored prophylaxis, we constructed a decoy-expressing lentiviral vector its effectiveness to the decoy-expressing AAV vectors. Lentiviral vector-transduced A549.ACE2 and CHME3.ACE2 cells expressed the decoy protein at a level similar to those of the AAV vectors (FIG. 13). Challenge to the cells showed that they were highly protected against infection by D614G and the Omicron BA.1, BA.2, BA.2.75, BA.4/5 and BQ1 pseudotyped viruses (FIG. 4A). Calculation of the MOI required to decrease infection by half, showed that BA.2 was the most resistant to neutralization, consistent with the results with the AAV vectors (FIG. 4A, below).

To determine whether the decoy-expressing lentiviral vectors could establish vectored prophylaxis, mice were injected i.v. or treated i.n. with the vector and after 7 days, challenged with WA1/2020 or Omicron BA.1 Virus loads in the lung were quantified 3-dpi (FIG. 4B, top). In mice treated i.v. or i.n. with decoy-expressing lentiviral vector and challenged with WA1/2020 SARS-COV-2, virus loads decreased nearly 4-logs, a level indistinguishable from uninfected control mice (FIG. 4B, bottom). A dose-response analysis in which mice were treated with decreasing amounts of the vector showed that for both routes of administration, 5×10⁵ I.U. was required to suppress the virus loads to undetectable levels (FIG. 14). The vector was also highly protective against Omicron BA.1, decreasing virus loads to nearly undetectable. To compare the protective effect of the vector with that of a therapeutic monoclonal antibody, mice were administered the potent neutralizing monoclonal antibody LY-CoV1404 by i.v. and i.n. routes prior to Omicron BA.1 infection. The results showed that the monoclonal antibody was effective by both routes although was somewhat less effective than i.v. lentiviral vector (FIG. 4B, bottom).

To test the durability of lentiviral vectored prophylaxis, mice were treated i.v. or i.n. with decoy-expressing or control GFP-expressing lentiviral vector and challenged 7, 30 and 60 days later with WA1/2020 SARS-COV-2. Virus loads were measured 3 days post-challenge. The mice were highly protected over the time-course (FIG. 4C). With increasing time post-treatment with the vector, viral load suppression became even stronger in mice that had been treated with the vector by the i.n. route.

To understand the basis of the long-lived protection provided by lentiviral vectored prophylaxis, mice were administered a luciferase-expressing lentiviral vector i.v. or i.n. and the level of vector expression over the 60-day time-course was determined by measuring luciferase activity in cell lysates prepared from different tissues. The results showed that i.v. administration of the vector resulted in the highest level expression in the spleen and about 10-fold lower expression in the lung that remained constant over the time-course (FIG. 4D). Low level expression was also detected in the liver, no expression was detected in the nasal tissue and trachea. Administration of the vector i.n. resulted in high level expression in the lung, moderate levels in the trachea (about 30-fold less on day 7) and undetectable levels in spleen and liver (FIG. 4E). Interestingly, the level of expression in the lung increased significantly at 60 days, a finding that would explain the increased viral load suppression at this time-point in mice treated by i.n. administration of the vector.

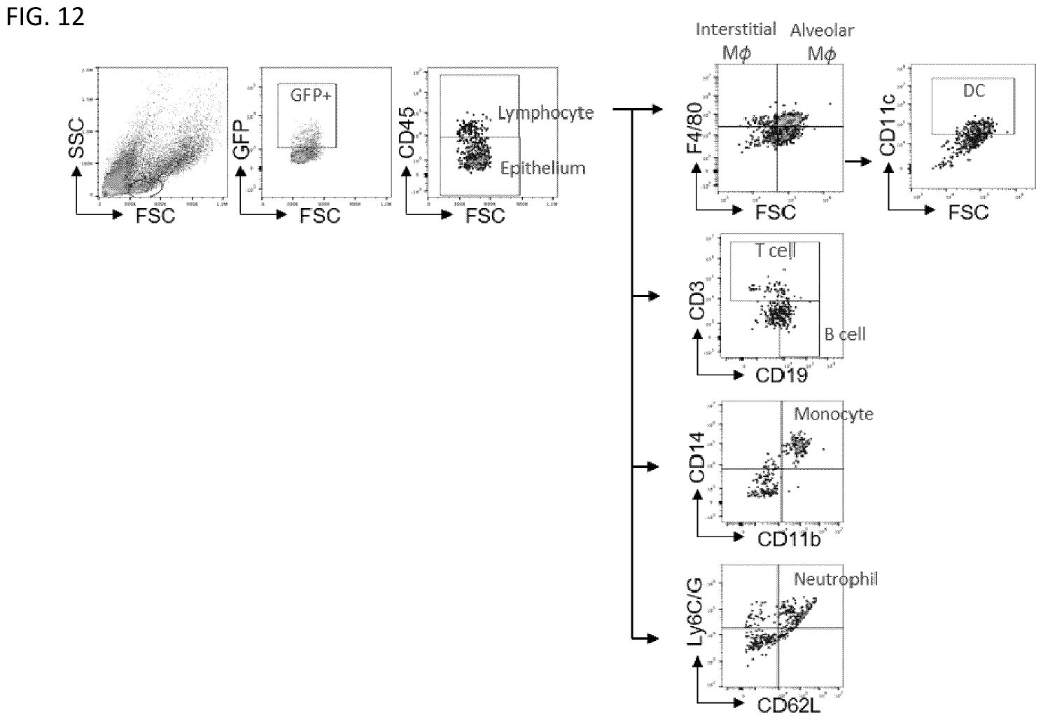

We next determined which cell types were transduced by AAV vectors. Mice were injected with AAV-GFP.Luc via i.n. injection, and three days post injection, the GFP+ population in the lung was analyzed by flow cytometry (FIG. 12). Of the non-epithelial cells, 11.5-14.4% were DCs, 11.2-14.1% were B cells, 10-11.2% were alveolar macrophages, and 20.9-30.6% were monocytes (FIG. 5A).

To determine which cell types were transduced by the lentiviral vector, mice were instilled i.n. with pLenti.GFP, and three days post-injection, the GFP+ population in the lung was analyzed by flow cytometry. Of the non-epithelial cells, 26.3% were DCs, 20.7% were B cells, 19.5% were alveolar macrophages, and 18.5% were monocytes (FIG. 5B).

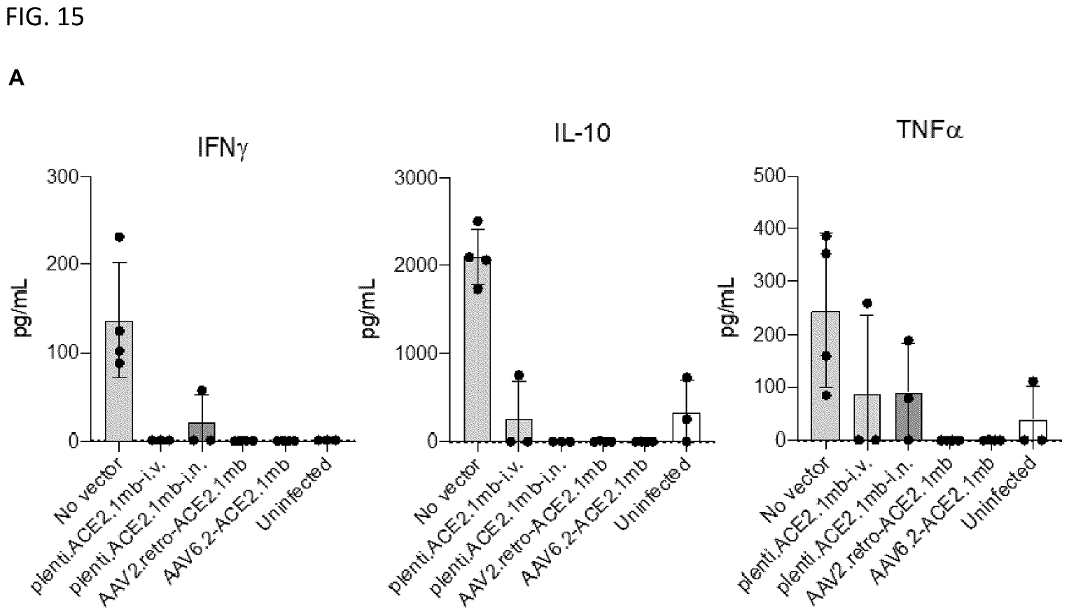

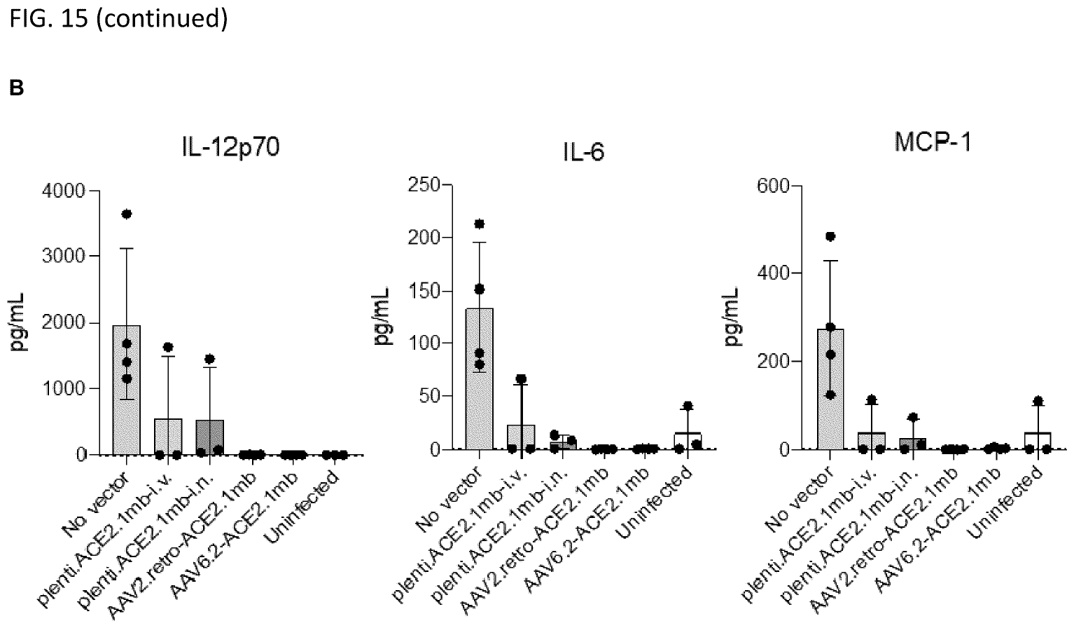

Treatment with the decoy-expressing lentiviral vector prevented the cytokine storm that is a hallmark of SARS-COV-2 infection as shown by the absence of an increase in IFNγ, TNFα, IL-6, IL-10, IL-12p70 and MCP-1 levels in the lung of mice treated 3 days post-challenge (FIG. 15).

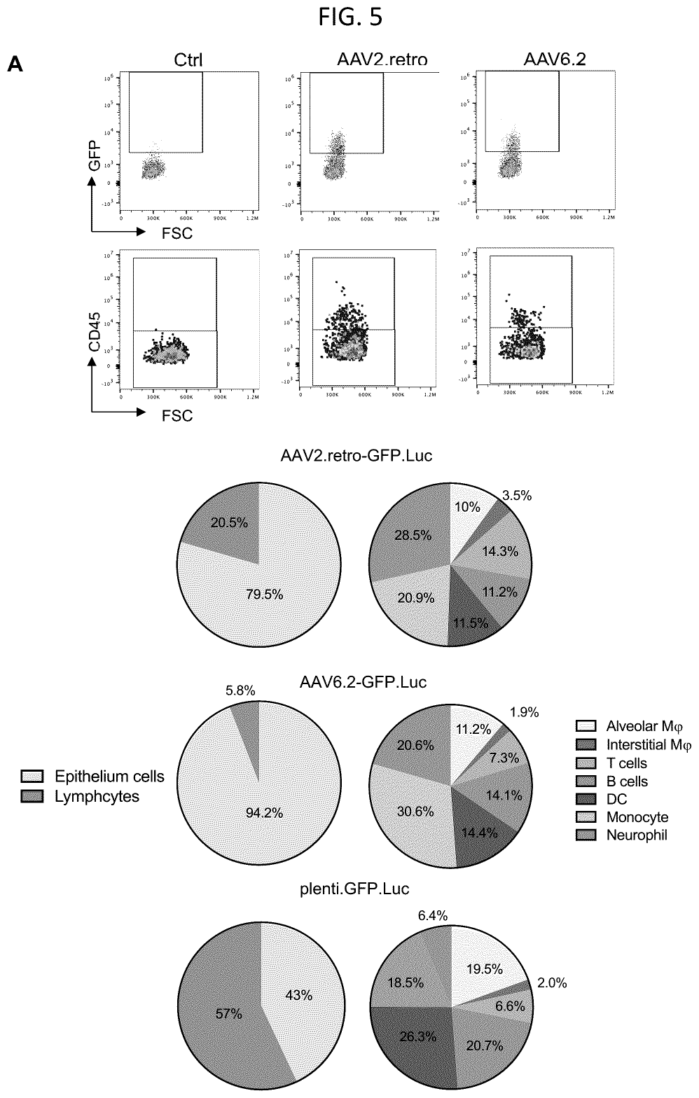

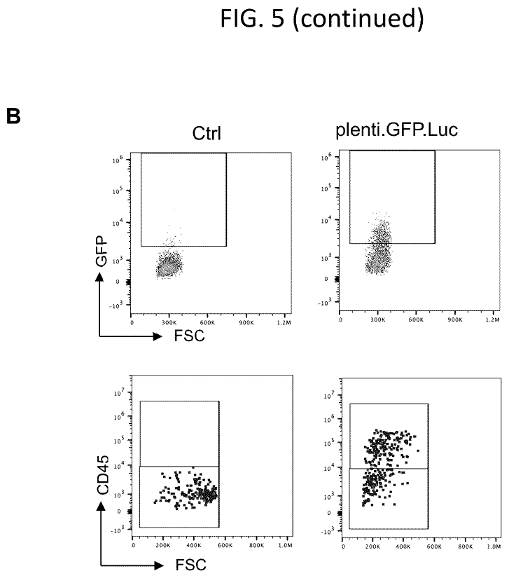

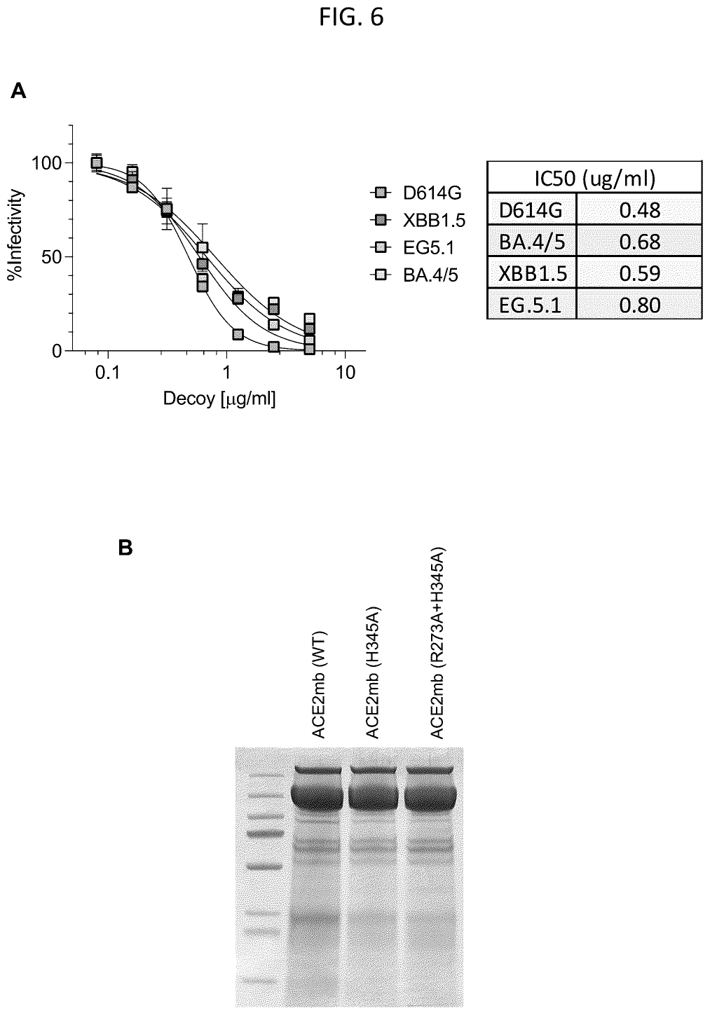

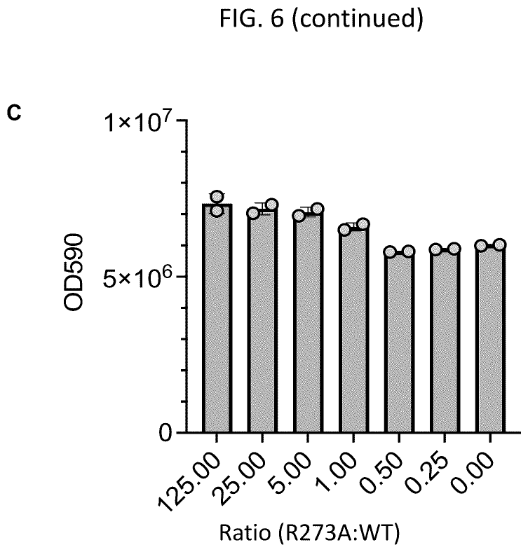

The decoy protein was able to prevent infect by pseudotyped viruses bearing the recent SARS-COV-2 variant spike proteins (FIG. 6A). The decoy protein with the H345A mutation was purified (FIG. 6B). The mutated decoy did not interfere with the function of catalytically active ACE2 as shown in a competition assay in which the mutated decoy was mixed in increasing amounts with the active protein and ACE2 catalytic activity was measured in an in vitro assay (FIG. 6B). The H345A mutated decoy was found to maintain some residual catalytic activity. The introduction of an R273A mutation completely inactivated the protein without affecting antiviral activity. The mutations ensure that the decoy protein will not have unwanted side-effects on blood pressure. The disclosure includes decoy protein with the H345A mutation, the R273A, mutation, and a combination thereof. In an embodiment, a described decoy protein consists of only one mutation, which may be the R273A mutation. In this regard, the 345 amino acid mutation did not fully inactivate catalytic activity whereas decoy protein with a R273A mutation was fully inactivated and maintained anti-viral activity.

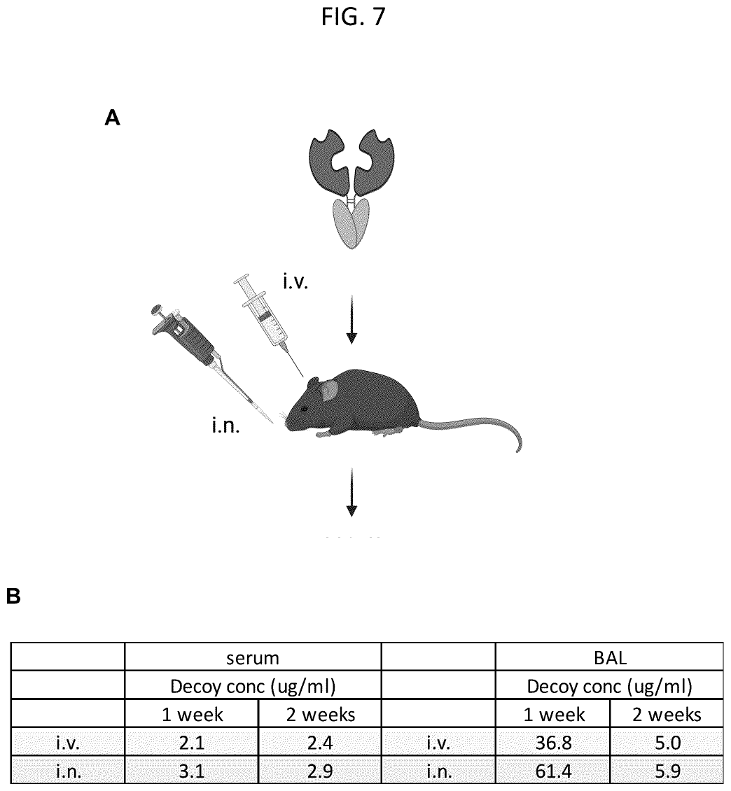

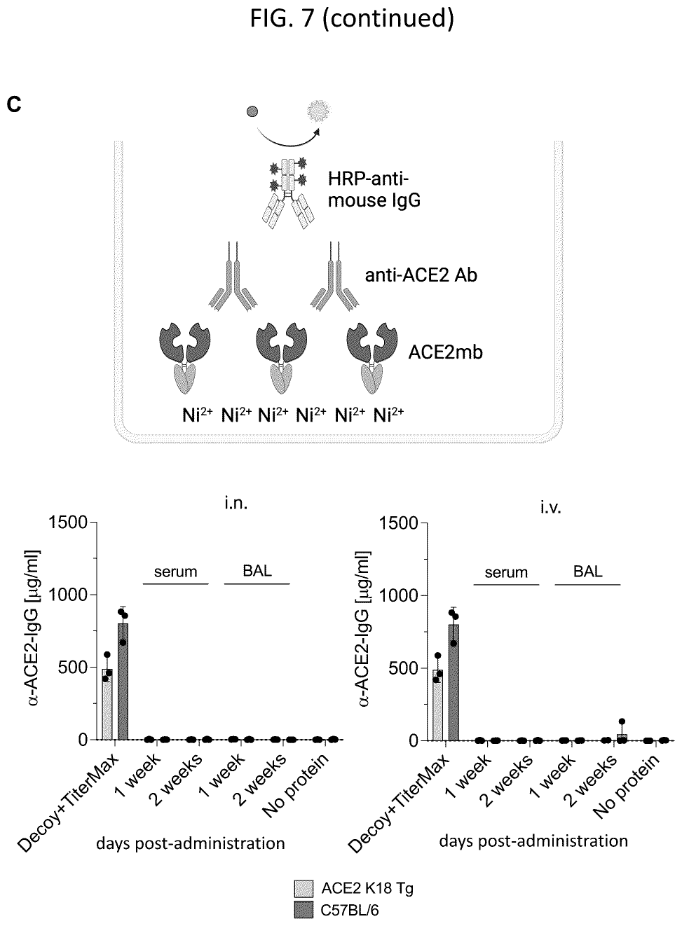

The decoy protein did not induce an unwanted anti-drug antibody in mice injected with the decoy protein (FIGS. 7A and C). Serum concentration of the decoy in the blood of mice and in the lungs was low, indicating the strong protective function of the decoy (FIG. 7B)

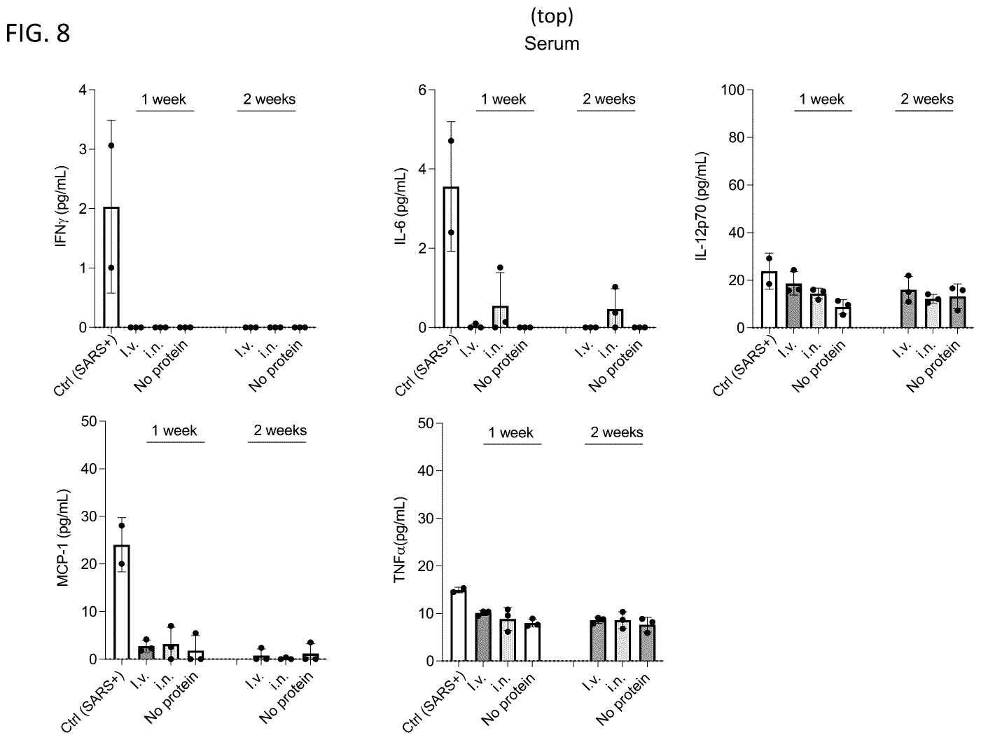

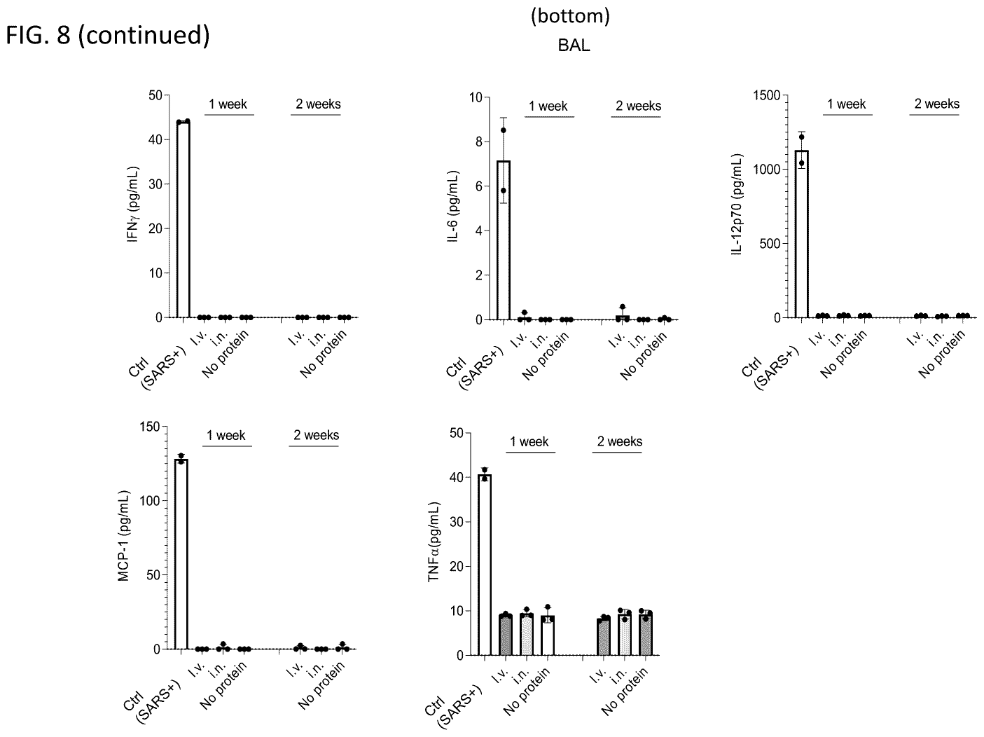

The decoy protein does not induce an inflammatory response in mice. (FIG. 8). Mice treated i.v. or i.n. with decoy protein did not show elevated levels of proinflammatory cytokines IFNγ, TNFα, IL-10, MCP-1 and IL-12p70.

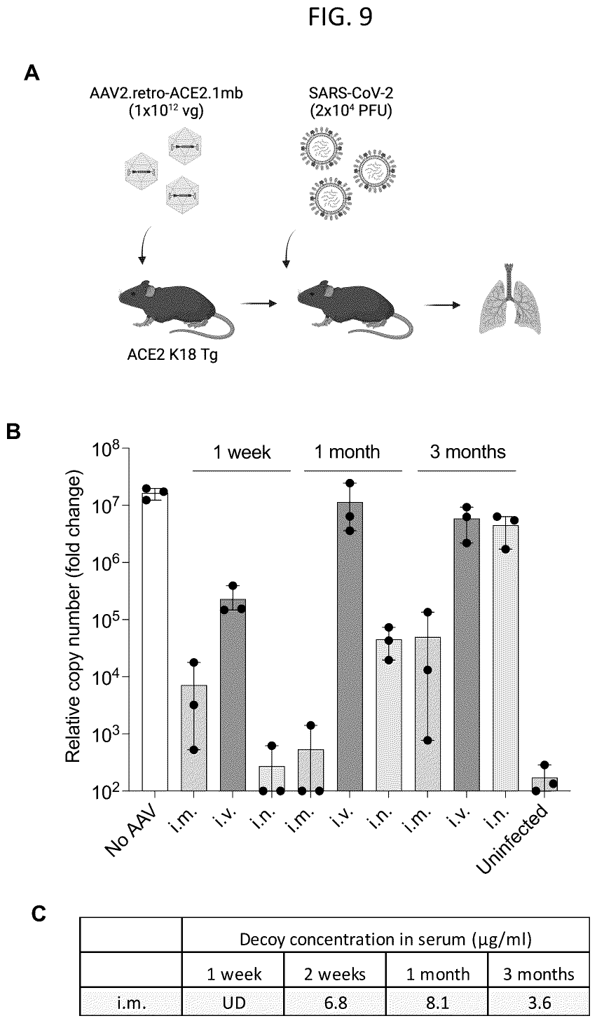

Mice treated by i.m. injection of decoy-expressing AAV2.retro were protected from SARS-COV-2 infection over 3 months (FIG. 9).

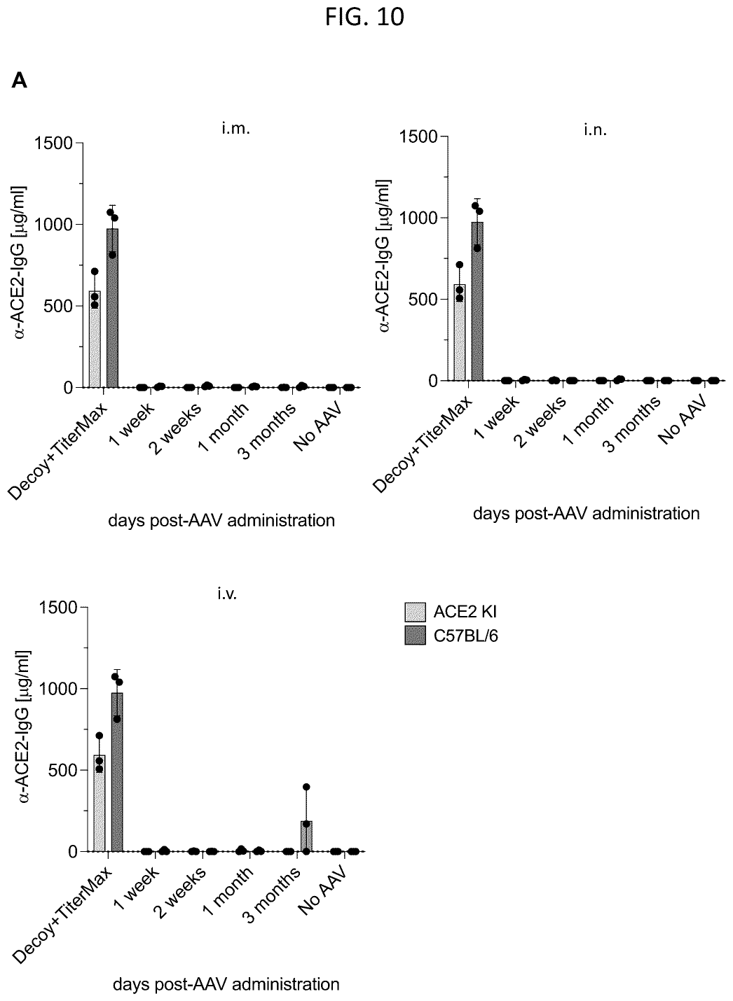

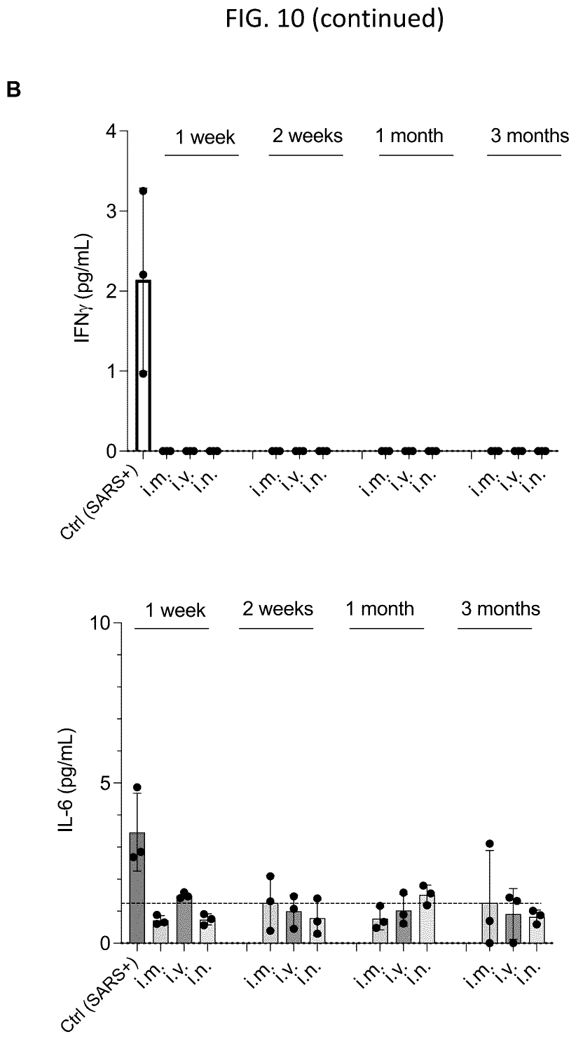

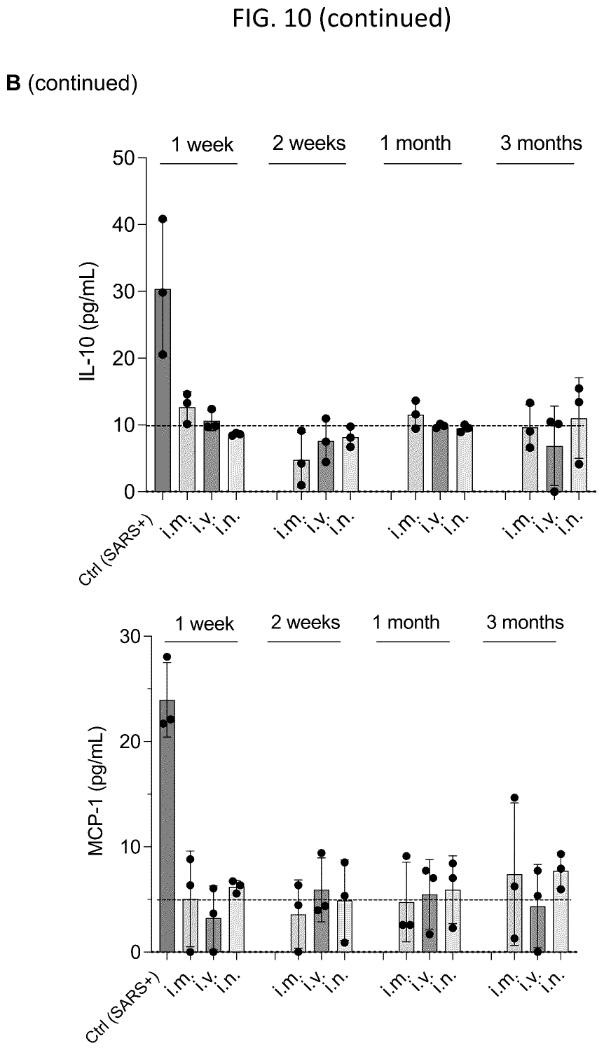

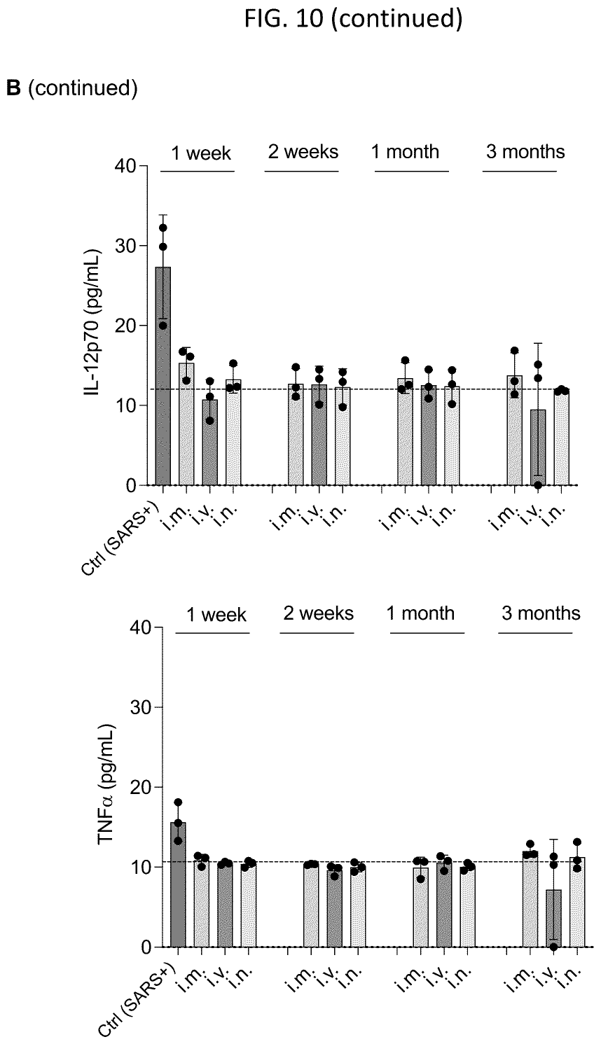

Mice treated with decoy-expressing AAV2.retro did not develop anti-drug antibody or induce proinflammatory cytokines (FIGS. 10A and B).

### Example 6

### Materials and Methods

### Cells

293T cell was cultured in DMEM/10% FBS. ACE2.CHME3, ACE2.A549 and ACE2.TMPRSS2. Vero E6 cells were cultured in the same medium with the addition of 1 μg/ml puromycin. ExpiCHO-S cells (Thermo Fisher Scientific) were grown at 37° C. under 8% CO₂ in suspension in ExpiCHO serum-free expression medium.

### Mice

C57BL/6 mice were from Taconic. Balb/c and hACE2 K18 mice [B6.Cg-Tg(K18-ACE2)₂Prlmn/J] were from The Jackson Laboratory. Animal use and care was approved by the NYU Langone Health Institutional Animal Care and Use Committee (#170304) according to the standards set by the Animal Welfare Act.

### Plasmids

The AAV-retro Rep/Cap2 (Addgene 81070), pAdDeltaF6 (Addgene 112867), Rep/Cap6 was (Addgene 110770) and pAAV-CAG-tdTomato were used to produce AAV vector stocks. To generate Rep/Cap6.2, an F129L mutation was introduced by overlap extension PCR and the amplicon was cloned into the EcoR-I and Nru-I sites of Rep/Cap6. To construct the GFP/nanoluciferase expression vector pAAV-GFP/NLuc, pAAV-CAG-ACE2.1mb-NLuc and pAAV-CAG-decoy, DNA fragments encoding GFP/Nluc and ACE2-NLuc were amplified by PCR and joined by overlap extension PCR using primers containing Kpn-I and EcoR-I sites. The resulting amplicons were cloned into Kpn-I and EcoR-I cleaved pAAV-CAG-tdTomato. The decoy expression vectors pcACE2.1mb have been previously described.

### AAV Vector Stocks

AAV vector stocks were produced by cotransfection of 293T cells with pAAV-CAG-decoy, FA6 and RapCap2 or Rep/Cap6.2 at a ratio of 25:25:30 by the calcium phosphate method. Virus-containing supernatant was harvested 2 days post-transfection and the virus was concentrated by ultracentrifugation at 4° C. for 16 hours at 30,000× g. The pellets were resuspended in PBS and concentrated by a factor of 500 on an Amicon Centrifugal filter. Virus titers were measured by RT-qPCR a primer pair and probe hybridizing to the AAV2 ITR sequences.

### SARS-COV-2 Virus Stocks

SARS-COV-2 WA1/2020 (BEI Resources, NR-52281) and Omicron BA.1 BA.2 (BEI Resources, NR-56781) and BA.5 virus (BEI Resources, NR-58616) stocks were prepared by infection of ACE2.TMPRSS2.Vero E6 cells at an MOI=0.05 (BEI Resources, NR-56781). Two hours post-infection, the input virus was removed and a day later, the virus-containing supernatant was filtered through a 0.45 μm filter and the virus was concentrated on an Amicon Ultra Centrifugal Filter Unit and stored in aliquots at −80° C.

### Recombinant Protein Purification

Protein purification was described previously. Briefly, ExpiCHO-S cells (6×10⁶/ml) were transfected with 400 μg pcACE2.1mb with ExpiFectamine. After 12 hours, ExpiCHO Enhancer and ExpiCHO Feed were added. After 4 days, the culture supernatant was harvested and passed over a 0.45 μm filter. The supernatant was passed on a 5 ml HiTrap Chelating column charged with nickel on an Akta FPLC (GE healthcare). The column was washed with buffer containing 20 mM Tris pH 8, 150 mM NaCl, 10 mM imidazole and the bound protein was then eluted in buffer containing 250 mM imidazole. The eluate was loaded onto a Superdex 200 size-exclusion column (GE healthcare) in running buffer containing 10 mM Tris pH 7.4, 150 mM NaCl.

### Decoy Pull-Down Assay

Cells (2×10⁵) were infected with AAV2.retro or AAV6.2-ACE2.1mb at MOI=0.5. The virus was removed after 1 day and the supernatant was harvested three days post-transfection. The decoy protein was pulled-down from the supernatant with 30 μl of nickel-nitrilotriacetic acid-agarose beads (QIAGEN) for 1 hour. The bound decoy was eluted in Laemmle loading buffer containing reducing agent and analyzed on an immunoblot probed with anti-His antibody and horseradish peroxidase (HRP)-conjugated goat anti-mouse lgG secondary antibody (Sigma-Aldrich). The signals were developed with Luminata Crescendo Western HRP Substrate (Millipore) and the signals were visualized on an iBright imaging system (Invitrogen).

### Pseudotype Neutralization

Spike protein-pseudotyped lentiviruses were generated in by transfection of 293T cells with packaging vector and spike protein expression vector as previously described. For the neutralization assay with ACE2.1mb protein, serially diluted decoy was incubated with pseudotyped virus (MOI=0.2) for 30 minutes at room temperature and then added to ACE2.CHME3 or ACE2.TMPRSS2.Vero E6 cells. At 2 days posy infection, luciferase activity was measured in an Envision 2103 microplate luminometer (PerkinElmer).

### Flow Cytometry

Mouse lung cells were blocked with anti-CD16/CD32 mAbs and stained with Alexa 700-anti-CD45, PerCP-Cy5.5-anti-F4/80, APC-Cy7-SiglecF, PE-Cy7-anti-CD11c, PE-Cy7-anti-CD19, APC-anti-CD3, Pacblue-anti-CD11b, PE-Cy5.5-anti-CD62L, APC-anti-CD14 and PE-Ly6C/Ly6G (Gr1) (all BioLegend). The cells were analyzed on an LSR-II flow cytometer and the data were analyzed with FlowJo software. Each cell types were classified as follows. Epithelium cells (CD45-), alveolar macrophage (CD45+, F4/80+, SiglecF+), interstitial macrophage (CD45+, F4/80+, SiglecF−), DC (CD45+, F4/80−, CD11c+), T cells (CD45+, CD3+), B cells (CD45+, CD19+), monocyte (CD45+, CD11b+, CD14+), neutrophil (CD45+, CD62L+, Ly6C/Ly6G+).

Proinflammatory cytokine assay Serum (10 μl) and lung from mice challenged with SARS-COV-2 WA1/2020 virus harvested 3-dpi, weighed and homogenized in an equal mass of PBS. The lysate was clarified by centrifugation for 5 minutes at 3000×g. The levels of IFN-γ, MCP-1, TNF-α, IL-10, IL-12 and IL-6 in 20 μl of lysate were measured by cytokine bead array using the BD Cytometric Bead Array Mouse Inflammation Kit (BD Biosciences).

### In Vivo and In Vitro Luciferase Assays

C57BL/6 or Balb/c mice were injected with AAV2.retro or AAV6.2-GFPNLuc or ACE2-NLuc (1×10¹² copies) via i.n. or i.e. injection. After 1-30 days of injection, the mice imaged on an IVIS Lumina III XR (PerkinElmer) with 100 μl 1:40 diluted Nano-GLO substrate (Nanolight). Alternatively, lung and brain were harvested and incubated with 100 μl 1:40 diluted Nano-GLO substrate for 30 second and imaged on an IVIS Lumina III XR. To measure the luciferase activity, the tissues were homogenized in lysing matrix D tubes by a FastPrep-24 5G homogenizer (MP Biomedicals). Organ lysates were mixed with Nano-Glo Luciferase Assay Reagent (Nanolight) and the activity was measured on an Envision 2103 plate reader (PerkinElmer).

### In Vitro AAV-Decoy Treatment

293T, CaCO2, ACE2.CHME3, ACE2.A549 and ACE2.TMPRSS2.Vero E6 cells (2×10⁵) were infected with AAV2.retro or AAV6.2-ACE2.1mb at MOI-0.5. After 1 day of infection, the virus was removed. After 1 day of incubation, the cells were infected with SARS-COV-2 at MOI=0.01. After 2 days of incubation, RNA was purified and virus load was measured by RT-qPCR.

Prophylaxis and Treatment of Mice with AAV-Decoy

For prophylaxis experiment, 6-8 weeks old hACE2-K18 Tg or Balb/c mice were anesthetized with isoflurane or ketamine-xylazine cocktail and injected 80 μl (i.v. or i.n.) or 40 μl (i.e.) (5×10¹² copies) of AAV2.retro or AAV6.2-decoy. After 1-30 days of infection, the mice were infected i.n. with 2.0×10⁴ PFU of SARS-COV-2 WA1/2020 (hACE2-K18 Tg) or Omicron BA.1 or BA.2 or BA.5 (Balb/c). At 2 days post infection (Omicron) or 3 days post infection (SARS-COV-2 WA1/2020), the mice were sacrificed and RNA was prepared from 200 μl of the lung or brain lysate using the Quick-RNA MiniPrep kit (Zymo Research). For treatment experiment, hACE2-K18 Tg were infected i.n. with 2.0×10⁴ PFU of SARS-COV-2 WA1/2020. After 0-48 hours infection, mice were injected i.v. with 80 μl (1×10¹² copies) of AAV2.retro or AAV6.2-decoy. Three days post infection (SARS-COV-2 WA1/2020), the mice were sacrificed and RNA was prepared from 200 μl of the lung or brain lysate using the Quick-RNA MiniPrep kit.

### Virus Loads

SARS-COV-2 subgenomic E RNA levels were measured by RT-qPCR with TaqMan probe as previously described. Briefly, lung RNA was mixed with TaqMan Fast Virus 1-step Master Mix (Applied Biosystems), 10 mM forward and reverse primers, and 2 mM probe. PCR was for 5 minutes at 50° C. followed by 95° C./20s and 40 cycles 95° C./3s, 60° C./30s). Data from tissue analyses were normalized to GAPDH. Virus load was determined by the 2-ΔΔCT method.

### Histology

Three days post SARS-COV-2 infection, the lung was harvested and fixed in 10% neutral buffered formalin for 72 hours at room temperature and then processed through graded ethanol, xylene and into paraffin in a Leica Peloris automated processor. Five-micron paraffin-embedded sections were deparaffinized and stained with hematoxylin (Leica, 3801575) and eosin (Leica, 3801619) on a Leica ST5020 automated histochemical strainer. Slides were scanned on at 40× on a Leica AT2 whole slide scanner and images transferred to the NYU Omero web-accessible image database.

### Statistical Analysis

Statistical significance was determined by Kruskal-Wallis test with post hoc Dunn's test. Significance was calculated based on two-sided testing and is shown in the figures as the mean ±SD with confidence intervals listed as *p<0.05, ** p<0.01, *** p<0.001, **** p<0.0001.

### Study Approval

Animal procedures were performed with the written approval of the NYU Animal Research Committee in accordance with all federal, state, and local guidelines.

### Discussion of Examples

The disclosure demonstrates in some embodiments that vectored prophylaxis using AAV or lentiviral vectors expressing a high affinity SARS-COV-2 a decoy receptor is highly effective. In mice treated with decoy-expressing AAV2.retro or AAV6.2 vector and later challenged with a high titered i.n. dose of WA1/2020 SARS-COV-2, virus was undetectable 3-dpi in the lungs using a highly sensitive RT-PCR assay, corresponding to a>10,000-fold decrease in virus load and the lung pathogenicity and decreased body weight characteristic of SARS-COV-2 infection in the mouse model was alleviated. The AAV vectors were most effective when administered by i.n. but were also effective by i.m. injection, suggesting that the decoy protein readily diffused away from the site of synthesis in the muscle to the lung and to other organs. Treatment with the decoy-expressing AAV vectors rapidly established protection that established protection that waned only slightly by 30-days post-treatment. The decoy-expressing lentiviral vector established a similarly high degree protection administered i.n. and was also effective through the i.v. route. Lentiviral vectored prophylaxis remained strong through the 60-day time course and appeared to intensify over-time as a result of increased expression from the vector in lung tissue over time as demonstrated using a luciferase-expressing reporter vector. Vectored prophylaxis was effective against all of the variants tested, including several of the Omicron subvariants. BA.2 was about 10-fold less-sensitive to neutralization by the decoy as compared to virus with the D614G spike protein, which may have been a result of its relative decreased affinity for the spike protein. More recent Omicron variant BA.5 was highly sensitive to the decoy.

There are over 20 AAV serotypes, each with unique tissue tropism. AAV6.2 is a variant of AAV6 that contain a single point mutation introduced to increase lung cell tropism. AAV2.retro is an AAV2 variant selected for retrograde transport in the CNS. Both vectors were effective for vectored prophylaxis administered i.n. AAV2.retro, which has not been reported to transduce cells of the respiratory system was, unexpectedly, somewhat more effective than AAV6.2.

Lentiviral vectors are currently being developed for several clinical applications including SARS-COV-2 vaccines. They have been generally used for the ex vivo transduction of cells in order to express therapeutic proteins. The vectors are stably expressed and are generally not subject to pre-existing immunity but their safety profile for direct injection purposes remains to be fully understood given their ability to integrate into the host cell genomic DNA (Mehrabadi et al., 2022; Mohanty et al., 2019; Sterner and Sterner, 2021).

Data presented herein support the use of vectored prophylaxis to provide long-lasting protection to immunocompromised individuals for whom vaccination is less effective. Until recently, the most effective protection available for such individuals was the AstraZeneca Evusheld cocktail, a mixture of two monoclonal antibodies formulated for slow release by intramuscular injection (Levin et al., 2022). However, both of the antibodies in the cocktail have significantly decreased neutralizing titers against the Omicron BA.1 and BA.2 subvariants (Iketani et al., 2022; Liu et al., 2022; Tada et al., 2022b; Zhou et al., 2022) and recent findings suggest that they may be inactive against the BQ1 and BA.2.75 subvariants that are currently increasing in prevalence. In contrast, the decoy maintained its effectiveness against AAV-based vectored prophylaxis could also be useful therapeutically. Delivery of the AAV vectors i.n. up to 24 hours post-infection infection caused a rapid decrease in virus load. While the therapy lost efficacy at later time points, the kinetics were similar to for the use of highly potent therapeutic monoclonal antibodies. Monoclonal antibodies have been found to lessen disease symptoms when given to patients several days post-infection and the same may apply to the use of decoy-expressing vectors.

Data presented herein also supports decoy receptor vectored prophylaxis in the case of a future pandemic spurred by zoonosis of a novel coronavirus. Species such as bats and pangolins harbor large numbers of coronaviruses with the ability to use human ACE2 (hACE2), would could serve as a reservoir of viruses with pandemic potential (Demogines et al., 2012; Wacharapluesadee et al., 2021). In the case of zoonosis of a coronavirus that used ACE-2 as a viral entry receptor, decoy-expressing vectors as described herein could be used as an off-the-shelf agent that would be available prior to the production of a new vaccine and that could rapidly establish protection without the need to induce an immune response. The approach could also be applied more broadly to viruses using receptors other than ACE-2, such as by identification of the novel receptor and the construction of a soluble form of the receptor to serve as a decoy. This would be the case if a novel SARS-COV-2 variants were to emerge that used an alternative receptor. However, such a variant has not emerged despite high selective pressure on the viral spike protein to alter the amino acid sequence of the spike protein RBD.

Representative and non-limiting examples of polynucleotides and amino acid sequences encompassed by the present disclosure are shown in the following Tables. The sequences may be modified as discussed in the Examples of this disclosure.

TABLE A

| | |
|---|---|
| S fragment A | CGATGCTCACGGATCCGCCACCATGTTCGT |
| (cDNA) | GTTTCTGGTGCTGCTGCCTCTGGTGTCCAG |
| | CCAGTGTGTGAACCTGACCACCAGAACACA |
| | GCTGCCTCCAGCCTACACCAACAGCTTTAC |
| | CAGAGGCGTGTACTACCCCGACAAGGTGTT |
| | CAGATCCAGCGTGCTGCACTCTACCCAGGA |
| | CCTGTTCCTGCCTTTCTTCAGCAACGTGAC |
| | CTGGTTCCACGCCATCCACGTGTCCGGCAC |
| | CAATGGCACCAAGAGATTCGACAACCCCGT |
| | GCTGCCCTTCAACGACGGGGTGTACTTTGC |
| | CAGCACCGAGAAGTCCAACATCATCAGAGG |
| | CTGGATCTTCGGCACCACACTGGACAGCAA |
| | GACCCAGAGCCTGCTGATCGTGAACAACGC |
| | CACCAACGTGGTCATCAAAGTGTGCGAGTT |
| | CCAGTTCTGCAACGACCCCTTCCTGGGCGT |
| | CTACTACCACAAGAACAACAAGAGCTGGAT |
| | GGAAAGCGAGTTCCGGGTGTACAGCAGCGC |
| | CAACAACTGCACCTTCGAGTACGTGTCCCA |
| | GCCTTTCCTGATGGACCTGGAAGGCAAGCA |
| | GGGCAACTTCAAGAACCTGCGCGAGTTCGT |
| | GTTCAAGAACATCGACGGCTACTTCAAGAT |
| | CTACAGCAAGCACACCCCTATCAACCTCGT |
| | GCGGGATCTGCCTCAGGGCTTCTCTGCTCT |
| | GGAACCCCTGGTGGATCTGCCCATCGGCAT |
| | CAACATCACCCGGTTTCAGACACTGCTGGC |
| | CCTGCACAGAAGCTACCTGACACCTGGCGA |
| | TAGCAGCAGCGGATGGACAGCTGGTGCCGC |
| | CGCTTACTATGTGGGCTACCTGCAGCCTAG |
| | AACCTTCCTGCTGAAGTACAACGAGAACGG |
| | CACCATCACCGACGCCGTGGATTGTGCTCT |
| | GGATCCTCTGAGCGAGACAAAGTGCACCCT |
| | GAAGTCCTTCACCGTGGAAAAGGGCATCTA |
| | CCAGACCAGCAACTTCCGGGTGCAGCCCAC |
| | CGAATCCATCGTGCGGTTCCCCAATATCAC |
| | CAATCTGTGCCCCTTCGGCGAGGTGTTCAA |
| | TGCCACCAGATTCGCCTCTGTGTACGCCTG |
| | GAACCGGAAGCGGATCAGCAATTGCGTGGC |
| | CGACTACTCCGTGCTGTACAACTCCGCCAG |
| | CTTCAGCACCTTCAAGTGCTACGGCGTGTC |
| | CCCTACCAAGCTGAACGACCTGTGCTTCAC |
| | AAACGTGTACGCCGACAGCTTCGTGATCCG |
| | GGGAGATGAAGTGCGGCAGATTGCCCCTGG |
| | ACAGACAGGCAAGATCGCCGACTACAACTA |
| | CAAGCTGCCCGACGACTTCACCGGCTGTGT |
| | GATTGCCTGGAACAGCAACAACCTGGACTC |
| | CAAAGTCGGCGGCAACTACAATTACCTGTA |
| | CCGGCTGTTCCGGAAGTCCAATCTGAAGCC |
| | CTTCGAGCGGGACATCTCCACCGAGATCTA |
| | TCAGGCCGGCAGCACCCCTTGTAACGGCGT |
| | GGAAGGCTTCAACTGCTACTTCCCACTGCA |
| | GTCCTACGGCTTTCAGCCCACAAATGGCGT |
| | GGGCTATCAGCCCTACAGAGTGGTGGTGCT |
| | GAGCTTCGAACTGCTGCATGCCCCTGCCAC |
| | AGTGTGCGGCCCTAAGAAAAGCACCAATCT |
| | CGTGAAGAACAAATGCGTGAACTTCAACTT |
| | CAACGGCCTGACCGGCACCGGCGTGCTGAC |
| | AGAGAGCAACAAGAAGTTCCTGCCATTCCA |
| | GCAGTTTGGCCGGGATATCGCCGATACCAC |
| | AGACGCCGTTAGAGATCCCCAGACACTGGA |
| | AATCCTGGACATCACCCCTTGCAGCTTCGG |
| | CGGAGTGTCTGTGATCACCCCTGGCACCAA |
| | CACCAGCAATCAGGTGGCAGTGCTGTACCA |
| | GGACGTGAACTGTACCGAAGTGCCCGTGGC |
| | CATTCACGCCGATCAGCTGACACCTACATG |
| | GCGGGTGTACTCCACCGGCTCCAATGTGTT |
| | TCAAACGCGTGCCGGCTGTCGAATTCATCG |
| | TCGACG (SEQ ID NO: 2) |
| S fragment B | GGCAGCAATGGAATTCTGTTTCAGACGCGT |
| (cDNA) | GCCGGCTGCCTGATTGGAGCCGAACACGTG |
| | AACAACAGCTACGAGTGCGACATCCCTATC |
| | GGAGCCGGCATCTGTGCCAGCTACCAGACA |
| | CAGACAAACAGCCCCAGACGGGCCAGATCT |
| | GTGGCCAGCCAGAGCATCATTGCCTACACC |
| | ATGTCTCTGGGCGCCGAGAACAGCGTGGCC |
| | TACAGCAACAACTCTATCGCTATCCCCACC |
| | AACTTCACCATCAGCGTGACCACCGAGATC |
| | CTGCCTGTGTCCATGACCAAGACCAGCGTG |
| | GACTGCACCATGTACATCTGCGGCGATAGC |
| | ACCGAGTGCAGCAACCTGCTGCTGCAGTAC |
| | GGCAGCTTCTGCACCCAGCTGAATAGAGCC |
| | CTGACCGGAATCGCCGTGGAACAGGACAAG |
| | AACACCCAAGAGGTGTTCGCCCAAGTGAAG |
| | CAGATCTACAAGACCCCTCCTATCAAGGAC |
| | TTCGGCGGCTTCAACTTCAGCCAGATTCTG |
| | CCCGATCCTAGCAAGCCCAGCAAGCGGAGC |
| | TTCATCGAGGACCTGCTGTTCAACAAAGTG |
| | ACCCTGGCCGACGCCGGCTTCATCAAGCAG |
| | TATGGCGATTGCCTGGGCGACATTGCCGCC |
| | AGGGATCTGATTTGCGCCCAGAAGTTTAAC |
| | GGCCTGACCGTGCTGCCTCCTCTGCTGACC |
| | GATGAGATGATCGCCCAGTACACATCTGCC |
| | CTGCTGGCCGGCACAATCACAAGCGGCTGG |
| | ACATTTGGAGCTGGCGCCGCTCTGCAGATC |
| | CCCTTTGCTATGCAGATGGCCTACCGGTTC |
| | AACGGCATCGGCGTGACCCAGAACGTGCTG |
| | TACGAGAACCAGAAGCTGATCGCCAACCAG |
| | TTCAACAGCGCCATCGGCAAGATCCAGGAC |
| | AGCCTGAGCAGTACAGCCAGCGCTCTGGGA |
| | AAGCTGCAGGACGTGGTCAACCAGAATGCC |
| | CAGGCTCTGAACACCCTGGTCAAGCAGCTG |
| | AGCAGCAACTTCGGCGCCATCAGCAGCGTG |
| | CTGAACGACATCCTGAGCCGCCTGGATAAG |
| | GTGGAAGCCGAGGTGCAGATCGACCGGCTG |
| | ATTACAGGCAGACTGCAGAGCCTGCAGACC |
| | TACGTGACACAGCAGCTGATCAGAGCCGCC |
| | GAGATTAGAGCCTCTGCCAATCTGGCCGCC |
| | ACCAAGATGTCTGAGTGTGTGCTGGGCCAG |
| | AGCAAGAGAGTGGACTTTTGCGGCAAGGGC |
| | TACCACCTGATGAGCTTCCCTCAGTCTGCT |
| | CCTCACGGCGTGGTGTTTCTGCACGTGACA |
| | TACGTGCCCGCTCAAGAGAAGAATTTCACC |
| | ACCGCTCCAGCCATCTGCCACGACGGCAAA |
| | GCCCACTTTCCTAGAGAAGGCGTGTTCGTG |
| | TCCAACGGCACCCATTGGTTCGTGACTCAG |
| | CGGAACTTCTACGAGCCCCAGATCATCACC |
| | ACCGACAACACCTTCGTGTCCGGCAACTGC |
| | GACGTCGTGATCGGCATCGTGAACAATACC |
| | GTGTACGACCCTCTGCAGCCCGAGCTGGAC |
| | AGCTTCAAAGAGGAACTGGACAAGTACTTC |
| | AAGAACCACACAAGCCCCGACGTGGACCTG |
| | GGCGATATCAGCGGAATCAATGCCAGCGTC |
| | GTGAACATCCAGAAAGAGATCGACAGACTG |
| | AACGAGGTGGCCAAGAACCTGAACGAGAGC |
| | CTGATCGACCTGCAAGAGCTGGGGAAGTAC |
| | GAGCAGTATATCAAGTGGCCCTGGTACATC |
| | TGGCTGGGCTTTATCGCCGGCCTGATTGCC |
| | ATCGTGATGGTCACAATCATGCTGTGCTGC |
| | ATGACCAGCTGTTGCAGCTGCCTGAAGGGC |
| | TGCTGTAGCTGTGGCTCCTGCTGCAAGTTC |
| | GACGAGGACGATTCTGAGCCCGTGCTGAAG |
| | GGCGTGAAGCTGCACTACACATACCCCTAC |
| | GACGTGCCCGACTACGCCTAACTCGAGCGT |
| | CAGCGTA(SEQ ID NO: 3) |
| ACE2 | ATGTCAAGCTCTTCCTGGCTCCTTCTCAGC |
| (cDNA) | CTTGTTGCTGTAACTGCTGCTCAGTCCACC |
| | ATTGAGGAACAGGCCAAGACATTTTTGGAC |
| | AAGTTTAACCACGAAGCCGAAGACCTGTTC |
| | TATCAAAGTTCACTTGCTTCTTGGAATTAT |
| | AACACCAATATTACTGAAGAGAATGTCCAA |
| | AACATGAATAATGCTGGGGACAAATGGTCT |
| | GCCTTTTTAAAGGAACAGTCCACACTTGCC |
| | CAAATGTATCCACTACAAGAAATTCAGAAT |
| | CTCACAGTCAAGCTTCAGCTGCAGGCTCTT |
| | CAGCAAAATGGGTCTTCAGTGCTCTCAGA |
| | AGACAAGAGCAAACGGTTGAACACAATTCT |
| | AAATACAATGAGCACCATCTACAGTACTGG |
| | AAAAGTTTGTAACCCAGATAATCCACAAGA |
| | ATGCTTATTACTTGAACCAGGTTTGAATGA |
| | AATAATGGCAAACAGTTTAGACTACAATGA |
| | GAGGCTCTGGGCTTGGGAAAGCTGGAGATC |
| | TGAGGTCGGCAAGCAGCTGAGGCCATTATA |
| | TGAAGAGTATGTGGTCTTGAAAAATGAGAT |
| | GGCAAGAGCAAATCATTATGAGGACTATGG |
| | GGATTATTGGAGAGGAGACTATGAAGTAAA |
| | TGGGGTAGATGGCTATGACTACAGCCGCGG |
| | CCAGTTGATTGAAGATGTGGAACATACCTT |
| | TGAAGAGATTAAACCATTATATGAACATCT |
| | TCATGCCTATGTGAGGGCAAAGTTGATGAA |
| | TGCCTATCCTTCCTATATCAGTCCAATTGG |
| | ATGCCTCCCTGCTCATTTGCTTGGTGATAT |
| | GTGGGGTAGATTTTGGACAAATCTGTACTC |
| | TTTGACAGTTCCCTTTGGACAGAAACCAAA |
| | CATAGATGTTACTGATGCAATGGTGGACCA |
| | GGCCTGGGATGCACAGAGAATATTCAAGGA |
| | GGCCGAGAAGTTCTTTGTATCTGTTGGTCT |
| | TCCTAATATGACTCAAGGATTCTGGGAAAA |
| | TTCCATGCTAACGGACCCAGGAAATGTTCA |
| | GAAAGCAGTCTGCCATCCCACAGCTTGGGA |
| | CCTGGGGAAGGGCGACTTCAGGATCCTTAT |
| | GTGCACAAAGGTGACAATGGACGACTTCCT |
| | GACAGCTCATCATGAGATGGGGCATATCCA |
| | GTATGATATGGCATATGCTGCACAACCTTT |
| | TCTGCTAAGAAATGGAGCTAATGAAGGATT |
| | CCATGAAGCTGTTGGGGAAATCATGTCACT |
| | TTCTGCAGCCACACCTAAGCATTTAAAATC |
| | CATTGGTCTTCTGTCACCCGATTTTCAAGA |
| | AGACAATGAAACAGAAATAAACTTCCTGCT |
| | CAAACAAGCACTCACGATTGTTGGGACTCT |
| | GCCATTTACTTACATGTTAGAGAAGTGGAG |
| | GTGGATGGTCTTTAAAGGGGAAATTCCCAA |
| | AGACCAGTGGATGAAAAAGTGGTGGGAGAT |
| | GAAGCGAGAGATAGTTGGGGTGGTGGAACC |
| | TGTGCCCCATGATGAAACATACTGTGACCC |
| | CGCATCTCTGTTCCATGTTTCTAATGATTA |
| | CTCATTCATTCGATATTACACAAGGACCCT |
| | TTACCAATTCCAGTTTCAAGAAGCACTTTG |
| | TCAAGCAGCTAAACATGAAGGCCCTCTGCA |
| | CAAATGTGACATCTCAAACTCTACAGAAGC |
| | TGGACAGAAACTGTTCAATATGCTGAGGCT |
| | TGGAAAATCAGAACCCTGGACCCTAGCATT |
| | GGAAAATGTTGTAGGAGCAAAGAACATGAA |
| | TGTAAGGCCACTGCTCAACTACTTTGAGCC |
| | CTTATTTACCTGGCTGAAAGACCAGAACAA |
| | GAATTCTTTTGTGGGATGGAGTACCGACTG |
| | GAGTCCATATGCAGACCAAAGCATCAAAGT |
| | GAGGATAAGCCTAAAATCAGCTCTTGGAGA |
| | TAAAGCATATGAATGGAACGACAATGAAAT |
| | GTACCTGTTCCGATCATCTGTTGCATATGC |
| | TATGAGGCAGTACTTTTTAAAAGTAAAAAA |
| | TCAGATGATTCTTTTTGGGGAGGAGGATGT |
| | GCGAGTGGCTAATTTGAAACCAAGAATCTC |
| | CTTTAATTTCTTTGTCACTGCACCTAAAAA |
| | TGTGTCTGATATCATTCCTAGAACTGAAGT |
| | TGAAAAGGCCATCAGGATGTCCCGGAGCCG |
| | TATCAATGATGCTTTCCGTCTGAATGACAA |
| | CAGCCTAGAGTTTCTGGGGATACAGCCAAC |
| | ACTTGGACCTCCTAACCAGCCCCCTGTTTC |
| | CATATGGCTGATTGTTTTTGGAGTTGTGAT |
| | GGGAGTGATAGTGGTTGGCATTGTCATCCT |
| | GATCTTCACTGGGATCAGAGATCGGAAGAA |
| | GAAAAATAAAGCAAGAAGTGGAGAAAATCC |
| | TTATGCCTCCATCGATATTAGCAAAGGAGA |
| | AAATAATCCAGGATTCCAAAACACTGATGA |
| | TGTTCAGACCTCCTTTTAG |
| | (SEQ ID NO: 4) |

TABLE B

| | |
|---|---|
| SARS-CoV-2 | MFVFLVLLPLVSSQCVNLTTRTQLPPAYTN |
| S-HA | SFTRGVYYPDKVFRSSVLHSTQDLFLPFFS |
| (amino acid) | NVTWFHAIHVSGTNGTKRFDNPVLPFNDGV |
| | YFASTEKSNIIRGWIFGTTLDSKTQSLLIV |
| | NNATNVVIKVCEFQFCNDPFLGVYYHKNNK |
| | SWMESEFRVYSSANNCTFEYVSQPFLMDLE |
| | GKQGNFKNLREFVFKNIDGYFKIYSKHTPI |
| | NLVRDLPQGFSALEPLVDLPIGINITRFQT |
| | LLALHRSYLTPGDSSSGWTAGAAAYYVGYL |
| | QPRTFLLKYNENGTITDAVDCALDPLSETK |
| | CTLKSFTVEKGIYQTSNFRVQPTESIVRFP |
| | NITNLCPFGEVFNATRFASVYAWNRKRISN |
| | CVADYSVLYNSASFSTFKCYGVSPTKLNDL |
| | CFTNVYADSFVIRGDEVRQIAPGQTGKIAD |
| | YNYKLPDDFTGCVIAWNSNNLDSKVGGNYN |
| | YLYRLFRKSNLKPFERDISTEIYQAGSTPC |
| | NGVEGFNCYFPLQSYGFQPTNGVGYQPYRV |
| | VVLSFELLHAPATVCGPKKSTNLVKNKCVN |
| | FNFNGLTGTGVLTESNKKFLPFQQFGRDIA |
| | DTTDAVRDPQTLEILDITPCSFGGVSVITP |
| | GTNTSNQVAVLYQDVNCTEVPVAIHADQLT |
| | PTWRVYSTGSNVFQTRAGCLIGAEHVNNSY |
| | ECDIPIGAGICASYQTQTNSPRRARSVASQ |
| | SIIAYTMSLGAENSVAYSNNSIAIPTNFTI |
| | SVTTEILPVSMTKTSVDCTMYICGDSTECS |
| | NLLLQYGSFCTQLNRALTGIAVEQDKNTQE |
| | VFAQVKQIYKTPPIKDFGGFNFSQILPDPS |
| | KPSKRSFIEDLLFNKVTLADAGFIKQYGDC |
| | LGDIAARDLICAQKFNGLTVLPPLLTDEMI |
| | AQYTSALLAGTITSGWTFGAGAALQIPFAM |
| | QMAYRFNGIGVTQNVLYENQKLIANQFNSA |
| | IGKIQDSLSSTASALGKLQDVVNQNAQALN |
| | TLVKQLSSNFGAISSVLNDILSRLDKVEAE |
| | VQIDRLITGRLQSLQTYVTQQLIRAAEIRA |
| | SANLAATKMSECVLGQSKRVDFCGKGYHLM |
| | SFPQSAPHGVVFLHVTYVPAQEKNFTTAPA |
| | ICHDGKAHFPREGVFVSNGTHWFVTQRNFY |
| | EPQIITTDNTFVSGNCDVVIGIVNNTVYDP |
| | LQPELDSFKEELDKYFKNHTSPDVDLGDIS |
| | GINASVVNIQKEIDRLNEVAKNLNESLIDL |
| | QELGKYEQYIKWPWYIWLGFIAGLIAIVMV |
| | TIMLCCMTSCCSCLKGCCSCGSCCKFDEDD |
| | SEPVLKGVKLHYTYPYDVPDYA (SEQ ID |
| | NO: 5) |
| SARS-CoV-2 | MFVFLVLLPLVSSQCVNLTTRTQLPPAYTN |
| Δ19 S | SFTRGVYYPDKVFRSSVLHSTQDLFLPFFS |
| (amino acid) | NVTWFHAIHVSGTNGTKRFDNPVLPFNDGV |
| | YFASTEKSNIIRGWIFGTTLDSKTQSLLIV |
| | NNATNVVIKVCEFQFCNDPFLGVYYHKNNK |
| | SWMESEFRVYSSANNCTFEYVSQPFLMDLE |
| | GKQGNFKNLREFVFKNIDGYFKIYSKHTPI |
| | NLVRDLPQGFSALEPLVDLPIGINITRFQT |
| | LLALHRSYLTPGDSSSGWTAGAAAYYVGYL |
| | QPRTFLLKYNENGTITDAVDCALDPLSETK |
| | CTLKSFTVEKGIYQTSNFRVQPTESIVRFP |
| | NITNLCPFGEVFNATRFASVYAWNRKRISN |
| | CVADYSVLYNSASFSTFKCYGVSPTKLNDL |
| | CFTNVYADSFVIRGDEVRQIAPGQTGKIAD |
| | YNYKLPDDFTGCVIAWNSNNLDSKVGGNYN |
| | YLYRLFRKSNLKPFERDISTEIYQAGSTPC |
| | NGVEGFNCYFPLQSYGFQPTNGVGYQPYRV |
| | VVLSFELLHAPATVCGPKKSTNLVKNKCVN |
| | FNFNGLTGTGVLTESNKKFLPFQQFGRDIA |
| | DTTDAVRDPQTLEILDITPCSFGGVSVITP |
| | GTNTSNQVAVLYQDVNCTEVPVAIHADQLT |
| | PTWRVYSTGSNVFQTRAGCLIGAEHVNNSY |
| | ECDIPIGAGICASYQTQTNSPRRARSVASQ |
| | SIIAYTMSLGAENSVAYSNNSIAIPTNFTI |
| | SVTTEILPVSMTKTSVDCTMYICGDSTECS |
| | NLLLQYGSFCTQLNRALTGIAVEQDKNTQE |
| | VFAQVKQIYKTPPIKDFGGFNFSQILPDPS |
| | KPSKRSFIEDLLFNKVTLADAGFIKQYGDC |
| | LGDIAARDLICAQKFNGLTVLPPLLTDEMI |
| | AQYTSALLAGTITSGWTFGAGAALQIPFAM |
| | QMAYRFNGIGVTQNVLYENQKLIANQFNSA |
| | IGKIQDSLSSTASALGKLQDVVNQNAQALN |
| | TLVKQLSSNFGAISSVLNDILSRLDKVEAE |
| | VQIDRLITGRLQSLQTYVTQQLIRAAEIRA |
| | SANLAATKMSECVLGQSKRVDFCGKGYHLM |
| | SFPQSAPHGVVFLHVTYVPAQEKNFTTAPA |
| | ICHDGKAHFPREGVFVSNGTHWFVTQRNFY |
| | EPQIITTDNTFVSGNCDVVIGIVNNTVYDP |
| | LQPELDSFKEELDKYFKNHTSPDVDLGDIS |
| | GINASVVNIQKEIDRLNEVAKNLNESLIDL |
| | QELGKYEQYIKWPWYIWLGFIAGLIAIVMV |
| | TIMLCCMTSCCSCLKGCCSCGSCC (SEQ |
| | ID NO: 6) |
| SARS-CoV-2 | MFVFLVLLPLVSSQCVNLTTRTQLPPAYTN |
| Δ19 | SFTRGVYYPDKVFRSSVLHSTQDLFLPFFS |
| S-HA | NVTWFHAIHVSGTNGTKRFDNPVLPFNDGV |
| (amino acid) | YFASTEKSNIIRGWIFGTTLDSKTQSLLIV |
| | NNATNVVIKVCEFQFCNDPFLGVYYHKNNK |
| | SWMESEFRVYSSANNCTFEYVSQPFLMDLE |
| | GKQGNFKNLREFVFKNIDGYFKIYSKHTPI |
| | NLVRDLPQGFSALEPLVDLPIGINITRFQT |
| | LLALHRSYLTPGDSSSGWTAGAAAYYVGYL |
| | QPRTFLLKYNENGTITDAVDCALDPLSETK |
| | CTLKSFTVEKGIYQTSNFRVQPTESIVRFP |
| | NITNLCPFGEVFNATRFASVYAWNRKRISN |
| | CVADYSVLYNSASFSTFKCYGVSPTKLNDL |
| | CFTNVYADSFVIRGDEVRQIAPGQTGKIAD |
| | YNYKLPDDFTGCVIAWNSNNLDSKVGGNYN |
| | YLYRLFRKSNLKPFERDISTEIYQAGSTPC |
| | NGVEGFNCYFPLQSYGFQPTNGVGYQPYRV |
| | VVLSFELLHAPATVCGPKKSTNLVKNKCVN |
| | FNFNGLTGTGVLTESNKKFLPFQQFGRDIA |
| | DTTDAVRDPQTLEILDITPCSFGGVSVITP |
| | GTNTSNQVAVLYQDVNCTEVPVAIHADQLT |
| | PTWRVYSTGSNVFQTRAGCLIGAEHVNNSY |
| | ECDIPIGAGICASYQTQTNSPRRARSVASQ |
| | SIIAYTMSLGAENSVAYSNNSIAIPTNFTI |
| | SVTTEILPVSMTKTSVDCTMYICGDSTECS |
| | NLLLQYGSFCTQLNRALTGIAVEQDKNTQE |
| | VFAQVKQIYKTPPIKDFGGFNFSQILPDPS |
| | KPSKRSFIEDLLFNKVTLADAGFIKQYGDC |
| | LGDIAARDLICAQKFNGLTVLPPLLTDEMI |
| | AQYTSALLAGTITSGWTFGAGAALQIPFAM |
| | QMAYRFNGIGVTQNVLYENQKLIANQFNSA |
| | IGKIQDSLSSTASALGKLQDVVNQNAQALN |
| | TLVKQLSSNFGAISSVLNDILSRLDKVEAE |
| | VQIDRLITGRLQSLQTYVTQQLIRAAEIRA |
| | SANLAATKMSECVLGQSKRVDFCGKGYHLM |
| | SFPQSAPHGVVFLHVTYVPAQEKNFTTAPA |
| | ICHDGKAHFPREGVFVSNGTHWFVTQRNFY |
| | EPQIITTDNTFVSGNCDVVIGIVNNTVYDP |
| | LQPELDSFKEELDKYFKNHTSPDVDLGDIS |
| | GINASVVNIQKEIDRLNEVAKNLNESLIDL |
| | QELGKYEQYIKWPWYIWLGFIAGLIAIVMV |
| | TIMLCCMTSCCSCLKGCCSCGSCCYPYDVP |
| | DYA (SEQ ID NO: 7) |
| SARS-CoV-2 | MFVFLVLLPLVSSQCVNLTTRTQLPPAYTN |
| D614G | SFTRGVYYPDKVFRSSVLHSTQDLFLPFFS |
| Δ19 S | NVTWFHAIHVSGTNGTKRFDNPVLPFNDGV |
| (amino acid) | YFASTEKSNIIRGWIFGTTLDSKTQSLLIV |
| | NNATNVVIKVCEFQFCNDPFLGVYYHKNNK |
| | SWMESEFRVYSSANNCTFEYVSQPFLMDLE |
| | GKQGNFKNLREFVFKNIDGYFKIYSKHTPI |
| | NLVRDLPQGFSALEPLVDLPIGINITRFQT |
| | LLALHRSYLTPGDSSSGWTAGAAAYYVGYL |
| | QPRTFLLKYNENGTITDAVDCALDPLSETK |
| | CTLKSFTVEKGIYQTSNFRVQPTESIVRFP |
| | NITNLCPFGEVFNATRFASVYAWNRKRISN |
| | CVADYSVLYNSASFSTFKCYGVSPTKLNDL |
| | CFTNVYADSFVIRGDEVRQIAPGQTGKIAD |
| | YNYKLPDDFTGCVIAWNSNNLDSKVGGNYN |
| | YLYRLFRKSNLKPFERDISTEIYQAGSTPC |
| | NGVEGENCYFPLQSYGFQPTNGVGYQPYRV |
| | VVLSFELLHAPATVCGPKKSTNLVKNKCVN |
| | FNFNGLTGTGVLTESNKKFLPFQQFGRDIA |
| | DTTDAVRDPQTLEILDITPCSFGGVSVITP |
| | GTNTSNQVAVLYQGVNCTEVPVAIHADQLT |
| | PTWRVYSTGSNVFQTRAGCLIGAEHVNNSY |
| | ECDIPIGAGICASYQTQTNSPRRARSVASQ |
| | SIIAYTMSLGAENSVAYSNNSIAIPTNFTI |
| | SVTTEILPVSMTKTSVDCTMYICGDSTECS |
| | NLLLQYGSFCTQLNRALTGIAVEQDKNTQE |
| | VFAQVKQIYKTPPIKDFGGFNFSQILPDPS |
| | KPSKRSFIEDLLFNKVTLADAGFIKQYGDC |
| | LGDIAARDLICAQKFNGLTVLPPLLTDEMI |
| | AQYTSALLAGTITSGWTFGAGAALQIPFAM |
| | QMAYRFNGIGVTQNVLYENQKLIANQFNSA |
| | IGKIQDSLSSTASALGKLQDVVNQNAQALN |
| | TLVKQLSSNFGAISSVLNDILSRLDKVEAE |
| | VQIDRLITGRLQSLQTYVTQQLIRAAEIRA |
| | SANLAATKMSECVLGQSKRVDFCGKGYHLM |
| | SFPQSAPHGVVFLHVTYVPAQEKNFTTAPA |
| | ICHDGKAHFPREGVFVSNGTHWFVTQRNFY |
| | EPQIITTDNTFVSGNCDVVIGIVNNTVYDP |
| | LQPELDSFKEELDKYFKNHTSPDVDLGDIS |
| | GINASVVNIQKEIDRLNEVAKNLNESLIDL |
| | QELGKYEQYIKWPWYIWLGFIAGLIAIVMV |
| | TIMLCCMTSCCSCLKGCCSCGSCC (SEQ |
| | ID NO: 8) |
| Soluble ACE2 | MSSSSWLLLSLVAVTAAQSTIEEQAKTFLD |
| (amino acid) | KFNHEAEDLFYQSSLASWNYNTNITEENVQ |
| | NMNNAGDKWSAFLKEQSTLAQMYPLQEIQN |
| | LTVKLQLQALQQNGSSVLSEDKSKRLNTIL |
| | NTMSTIYSTGKVCNPDNPQECLLLEPGLNE |
| | IMANSLDYNERLWAWESWRSEVGKQLRPLY |
| | EEYVVLKNEMARANHYEDYGDYWRGDYEVN |
| | GVDGYDYSRGQLIEDVEHTFEEIKPLYEHL |
| | HAYVRAKLMNAYPSYISPIGCLPAHLLGDM |
| | WGRFWTNLYSLTVPFGQKPNIDVTDAMVDQ |
| | AWDAQRIFKEAEKFFVSVGLPNMTQGFWEN |
| | SMLTDPGNVQKAVCHPTAWDLGKGDFRILM |
| | CTKVTMDDFLTAHHEMGHIQYDMAYAAQPF |
| | LLRNGANEGFHEAVGEIMSLSAATPKHLKS |
| | IGLLSPDFQEDNETEINFLLKQALTIVGTL |
| | PFTYMLEKWRWMVFKGEIPKDQWMKKWWEM |
| | KREIVGVVEPVPHDETYCDPASLFHVSNDY |
| | SFIRYYTRTLYQFQFQEALCQAAKHEGPLH |
| | KCDISNSTEAGQKLFNMLRLGKSEPWTLAL |
| | ENVVGAKNMNVRPLLNYFEPLFTWLKDQNK |
| | NSFVGWSTDWSPYADQSIKVRISLKSALGD |
| | RAYEWNDNEMYLFRSSVAYAMRQYFLKVKN |
| | QMILFGEEDVRVANLKPRISFNFFVTAPKN |
| | VSDIIPRTEVEKAIRMSRSRINDAFRLNDN |
| | SLEFLGIQPTLGPPNQPPVSHHHHHHHH |
| | (SEQ ID NO: 9) |
| ACE2- | MSSSSWLLLSLVAVTAAQSTIEEQAKTFLD |
| microbody | KFNHEAEDLFYQSSLASWNYNTNITEENVQ |
| (amino acid) | NMNNAGDKWSAFLKEQSTLAQMYPLQEIQN |
| | LTVKLQLQALQQNGSSVLSEDKSKRLNTIL |
| | NTMSTIYSTGKVCNPDNPQECLLLEPGLNE |
| | IMANSLDYNERLWAWESWRSEVGKQLRPLY |
| | EEYVVLKNEMARANHYEDYGDYWRGDYEVN |
| | GVDGYDYSRGQLIEDVEHTFEEIKPLYEHL |
| | HAYVRAKLMNAYPSYISPIGCLPAHLLGDM |
| | WGRFWTNLYSLTVPFGQKPNIDVTDAMVDQ |
| | AWDAQRIFKEAEKFFVSVGLPNMTQGFWEN |
| | SMLTDPGNVQKAVCHPTAWDLGKGDFRILM |
| | CTKVTMDDFLTAHHEMGHIQYDMAYAAQPF |
| | LLRNGANEGFHEAVGEIMSLSAATPKHLKS |
| | IGLLSPDFQEDNETEINFLLKQALTIVGTL |
| | PFTYMLEKWRWMVFKGEIPKDQWMKKWWEM |
| | KREIVGVVEPVPHDETYCDPASLFHVSNDY |
| | SFIRYYTRTLYQFQFQEALCQAAKHEGPLH |
| | KCDISNSTEAGQKLFNMLRLGKSEPWTLAL |
| | ENVVGAKNMNVRPLLNYFEPLFTWLKDQNK |
| | NSFVGWSTDWSPYADQSIKVRISLKSALGD |
| | RAYEWNDNEMYLFRSSVAYAMRQYFLKVKN |
| | QMILFGEEDVRVANLKPRISFNFFVTAPKN |
| | VSDIIPRTEVEKAIRMSRSRINDAFRLNDN |
| | SLEFLGIQPTLGPPNQPPVSEPKSCDKTHT |
| | CPPCGGGSSGGGSGGQPREPQVYTLPPSRE |
| | EMTKNQVSLTCLVKGFYPSDIAVEWESNGQ |
| | PENNYKTTPPVLDSDGSFFLYSKLTVDKSR |
| | WQQGNVFSCSVMHEALHNHYTQKSLSLSPG |
| | KHHHHHHHH (SEQ ID NO: 10) |
| ACE2.H345A- | MSSSSWLLLSLVAVTAAQSTIEEQAKTFLD |
| microbody | KFNHEAEDLFYQSSLASWNYNTNITEENVQ |
| (amino acid) | NMNNAGDKWSAFLKEQSTLAQMYPLQEIQN |
| | LTVKLQLQALQQNGSSVLSEDKSKRLNTIL |
| | NTMSTIYSTGKVCNPDNPQECLLLEPGLNE |
| | IMANSLDYNERLWAWESWRSEVGKQLRPLY |
| | EEYVVLKNEMARANHYEDYGDYWRGDYEVN |
| | GVDGYDYSRGQLIEDVEHTFEEIKPLYEHL |
| | HAYVRAKLMNAYPSYISPIGCLPAHLLGDM |
| | WGRFWTNLYSLTVPFGQKPNIDVTDAMVDQ |
| | AWDAQRIFKEAEKFFVSVGLPNMTQGFWEN |
| | SMLTDPGNVQKAVCAPTAWDLGKGDFRILM |
| | CTKVTMDDFLTAHHEMGHIQYDMAYAAQPF |
| | LLRNGANEGFHEAVGEIMSLSAATPKHLKS |
| | IGLLSPDFQEDNETEINFLLKQALTIVGTL |
| | PFTYMLEKWRWMVFKGEIPKDQWMKKWWEM |
| | KREIVGVVEPVPHDETYCDPASLFHVSNDY |
| | SFIRYYTRTLYQFQFQEALCQAAKHEGPLH |
| | KCDISNSTEAGQKLFNMLRLGKSEPWTLAL |
| | ENVVGAKNMNVRPLLNYFEPLFTWLKDQNK |
| | NSFVGWSTDWSPYADQSIKVRISLKSALGD |
| | RAYEWNDNEMYLFRSSVAYAMRQYFLKVKN |
| | QMILFGEEDVRVANLKPRISFNFFVTAPKN |
| | VSDIIPRTEVEKAIRMSRSRINDAFRLNDN |
| | SLEFLGIQPTLGPPNQPPVSEPKSCDKTHT |
| | CPPCGGGSSGGGSGGQPREPQVYTLPPSRE |
| | EMTKNQVSLTCLVKGFYPSDIAVEWESNGQ |
| | PENNYKTTPPVLDSDGSFFLYSKLTVDKSR |
| | WQQGNVFSCSVMHEALHNHYTQKSLSLSPG |
| | KHHHHHHHH (SEQ ID NO: 11) |

References—this reference listing is not an indication that any reference is material to patentability:
ADDIN EN.REFLIST Adhikary, P., Kandel, S., Mamani, U.-F., Mustafa, B., Hao, S., Qiu, J., Fetse, J., Liu, Y., Ibrahim, N. M., Li, Y., et al. Discovery of Small Anti-ACE2 Peptides to Inhibit SARS-COV-2 Infectivity. Advanced Therapeutics n/a, 2100087.
Baum, A., Fulton, B. O., Wloga, E., Copin, R., Pascal, K. E., Russo, V., Giordano, S., Lanza, K., Negron, N., Ni, M., et al. (2020). Antibody cocktail to SARS-COV-2 spike protein prevents rapid mutational escape seen with individual antibodies. Science 369, 1014-1018.
Cao, L., Goreshnik, I., Coventry, B., Case, J. B., Miller, L., Kozodoy, L., Chen, R. E., Carter, L., Walls, A. C., Park, Y.-J., et al. (2020). De novo design of picomolar SARS-COV-2 miniprotein inhibitors. Science 370, 426-431.
Demogines, A., Farzan, M., and Sawyer, S. L. (2012). Evidence for ACE2-Utilizing Coronaviruses (CoVs) Related to Severe Acute Respiratory Syndrome CoV in Bats. Journal of Virology 86, 6350-6353.
Galmiche, S., Luong Nguyen, L. B., Tartour, E., de Lamballerie, X., Wittkop, L., Loubet, P., and Launay, O. (2022). Immunological and clinical efficacy of COVID-19 vaccines in immunocompromised populations: a systematic review. Clin Microbiol Infect 28, 163-177
Gardner, M. R., Kattenhorn, L. M., Kondur, H. R., von Schaewen, M., Dorfman, T., Chiang, J. J., Haworth, K. G., Decker, J. M., Alpert, M. D., Bailey, C. C., et al. (2015). AAV-expressed eCD4-Ig provides durable protection from multiple SHIV challenges. Nature 519, 87-91.
Gershon, D. (1996). Genentech sheds gp120 vaccine. Nature Medicine 2, 370-371.
Han, Y., and Král, P. (2020). Computational Design of ACE2-Based Peptide Inhibitors of SARS-COV-2. ACS Nano 14, 5143-5147.
Hansel, T. T., Kropshofer, H., Singer, T., Mitchell, J. A., and George, A. J. (2010). The safety and side effects of monoclonal antibodies. Nat Rev Drug Discov 9, 325-338.
Hansen, J., Baum, A., Pascal, K. E., Russo, V., Giordano, S., Wloga, E., Fulton, B. O., Yan, Y., Koon, K., Patel, K., et al. (2020). Studies in humanized mice and convalescent humans yield a SARS-COV-2 antibody cocktail. Science 369, 1010-1014.
Hodges, T. L., Kahn, J. O., Kaplan, L. D., Groopman, J. E., Volberding, P. A., Amman, A. J., Arri, C. J., Bouvier, L. M., Mordenti, J., and Izu, A. E. (1991). Phase 1 study of recombinant human CD4-immunoglobulin G therapy of patients with AIDS and AIDS-related complex. Antimicrobial Agents and Chemotherapy 35, 2580-2586.
Johnson, P. R., Schnepp, B. C., Zhang, J., Connell, M. J., Greene, S. M., Yuste, E., Desrosiers, R. C., and Clark, K. R. (2009). Vector-mediated gene transfer engenders long-lived neutralizing activity and protection against SIV infection in monkeys. Nat Med 15, 901-906.
Karoyan, P., Vieillard, V., Gómez-Morales, L., Odile, E., Guihot, A., Luyt, C.-E., Denis, A., Grondin, P., and Lequin, O. (2021). Human ACE2 peptide-mimics block SARS-COV-2 pulmonary cells infection. Communications Biology 4, 197.
Larue, R. C., Xing, E., Kenney, A. D., Zhang, Y., Tuazon, J. A., Li, J., Yount, J. S., Li, P.-K., and Sharma, A. (2021). Rationally Designed ACE2-Derived Peptides Inhibit SARS-COV-2. Bioconjugate Chemistry 32, 215-223.
Lee, A., Wong, S. Y., Chai, L. Y. A., Lee, S. C., Lee, M. X., Muthiah, M. D., Tay, S. H., Teo, C. B., Tan, B. K. J., Chan, Y. H., et al. (2022). Efficacy of covid-19 vaccines in immunocompromised patients: systematic review and meta-analysis. BMJ 376, e068632.
Levin, M. J., Ustianowski, A., De Wit, S., Launay, O., Avila, M., Templeton, A., Yuan, Y., Seegobin, S., Ellery, A., Levinson, D. J., et al. (2022). Intramuscular AZD7442 (Tixagevimab-Cilgavimab) for Prevention of Covid-19. N Engl J Med.
Limberis, M. P., Vandenberghe, L. H., Zhang, L., Pickles, R. J., and Wilson, J. M. (2009). Transduction efficiencies of novel AAV vectors in mouse airway epithelium in vivo and human ciliated airway epithelium in vitro. Mol Ther 17, 294-301.
Liu, L., Iketani, S., Guo, Y., Chan, J. F. W., Wang, M., Liu, L., Luo, Y., Chu, H., Huang, Y., Nair, M. S., et al. (2022). Striking antibody evasion manifested by the Omicron variant of SARS-COV-2. Nature 602, 676-681.
Morgan, D. C., Morris, C., Mahindra, A., Blair, C. M., Tejeda, G., Herbert, I., Turnbull, M. L., Lieber, G., Willett, B. J., Logan, N., et al. Stapled ACE2 peptidomimetics designed to target the SARS-COV-2 spike protein do not prevent virus internalization. Peptide Science n/a, e24217.
Planas, D., Saunders, N., Maes, P., Guivel-Benhassine, F., Planchais, C., Buchrieser, J., Bolland, W.-H., Porrot, F., Staropoli, I., Lemoine, F., et al. (2021). Considerable escape of SARS-COV-2 Omicron to antibody neutralization. Nature.
Spitsin, S., Schnepp, B. C., Connell, M. J., Liu, T., Dang, C. M., Pappa, V., Tustin, R., Kinder, A., Johnson, P. R., and Douglas, S. D. (2020). Protection against SIV in Rhesus Macaques Using Albumin and CD4-Based Vector-Mediated Gene Transfer. Molecular therapy Methods & clinical development 17, 1088-1096.
Tada, T., Norton, T. D., Leibowitz, R., and Landau, N. R. (2022). Directly injected lentiviral vector-based T cell vaccine protects mice against acute and chronic viral infection. JCI Insight.
Tervo, D. G., Hwang, B. Y., Viswanathan, S., Gaj, T., Lavzin, M., Ritola, K. D., Lindo, S., Michael, S., Kuleshova, E., Ojala, D., et al. (2016). A Designer AAV Variant Permits Efficient Retrograde Access to Projection Neurons. Neuron 92, 372-382.
VanBlargan, L. A., Errico, J. M., Halfmann, P. J., Zost, S. J., Crowe, J. E., Jr., Purcell, L. A., Kawaoka, Y., Corti, D., Fremont, D. H., and Diamond, M. S. (2022). An infectious SARS-CoV-2 B.1.1.529 Omicron virus escapes neutralization by therapeutic monoclonal antibodies. Nat Med.
Wacharapluesadee, S., Tan, C. W., Maneeorn, P., Duengkae, P., Zhu, F., Joyjinda, Y., Kaewpom, T., Chia, W. N., Ampoot, W., Lim, B. L., et al. (2021). Evidence for SARS-COV-2 related coronaviruses circulating in bats and pangolins in Southeast Asia. Nature Communications 12, 972.
Zhang, Y., Wang, J., Li, J., Chen, Y., Sun, J., Lu, Z., Li, Y., and Liu, T. (2022). Functional analysis of mutations endowing rAAV2-retro with retrograde tracing capacity. Neurosci Lett 784, 136746.
Zhou, H., Tada, T., Dcosta, B. M., and Landau, N. R. (2022). Neutralization of SARS-COV-2 Omicron BA.2 by Therapeutic Monoclonal Antibodies. bioRxiv, 2022.2002.2015.480166.

What is claimed is:

1. A polypeptide comprising an angiotensin-converting enzyme 2 (ACE2) ectodomain and a segment of an immunoglobulin Fc that is not an intact Fc region.

2. The polypeptide of claim 1, wherein the ACE2 ectodomain is enzymatically inactivated by a mutation of an amino acid in an ACE2 catalytically active site.

3. The polypeptide of claim 2, wherein the Fc comprises an Fc IgG-CH3.

4. The polypeptide of claim 3, wherein the lgG-CH3 is a human IgG-CH3.

5. A method for prophylaxis or therapy for a Coronavirus infection comprising introducing into an individual in need thereof an expression vector encoding a polypeptide of claim 1.

6. The method of claim 5, wherein the expression vector is a lentiviral expression vector or an adenoviral expression vector.

7. The method of claim 6, wherein the expression vector is an adeno-associated viral vector.

8. The method of claim 7, wherein the ACE2 ectodomain is enzymatically inactivated by a mutation of an amino acid in an ACE2 catalytically active site.

9. The method of claim 8, wherein the Fc comprises an Fc IgG-CH3.

10. The method of claim 8, wherein the lgG-CH3 is a human IgG-CH3.

11. The method of claim 10, wherein the administration is a prophylactic administration.

12. The method of claim 10, wherein the administration is a therapeutic administration.

13. The method of claim 11, wherein the administration is an intranasal or intramuscular administration.

14. The method of claim 12, wherein the administration is an intranasal or intramuscular administration.

15. The method of claim 8, wherein the Coronavirus infection is a SARS-COV-2 infection.

16. The method of claim 9, wherein the Coronavirus infection is a SARS-COV-2 infection.

17. The method of claim 10, wherein the Coronavirus infection is a SARS-COV-2 infection.

18. The method of claim 11, wherein the Coronavirus infection is a SARS-COV-2 infection.

19. The method of claim 12, wherein the Coronavirus infection is a SARS-COV-2 infection.

20. The method of claim 13, wherein the Coronavirus infection is a SARS-COV-2 infection.
